(19) United States
(12) Patent Application Publication
(10) Pub. No.: US 2008/0274456 A1
(43) Pub. Date: Nov. 6, 2008

(54) Methods and Compositions for Modifying Gene Regulation and Dna Damage in Ageing

(76) Inventors: Bruce Yankner, Newton, MA (US); Tao Lu, Brookline, MA (US)

Correspondence Address: NUTTER MCCLENNEN & FISH LLP, WORLD TRADE CENTER WEST, 155 SEAPORT BOULEVARD, BOSTON, MA, 02210-2604, US

(21) Appl. No.: 11/629,223

(22) PCT Filed: Jun. 9, 2005
(86) PCT No.: PCT/US05/20159

§ 371 Date: Jul. 16, 2008

Related U.S. Application Data
(60) Provisional application No. 60/582,329, filed on Jun. 9, 2004.

(51) Int. Cl. C12Q 1/68

(52) U.S. Cl. 435/6

(57) ABSTRACT

The invention relates to gene regulation in ageing, and age-related cognitive decline. The invention, in particular relates to methods for screening a subject for a propensity to develop diseases associated with oxidative stress, and for age-related conditions, by examining the up-regulation and/or down-regulation of at least one gene associated within the central nervous system.

### PRIORITY INFORMATION

This application claims the benefit of priority of U.S. Ser. No. 60/582,329, filed Jun. 9, 2004, entitled, “Methods and Compositions For Modifying Gene Regulation and DNA Damage in Ageing,” the teachings of which are incorporated herein by reference.

### BACKGROUND OF THE INVENTION

The invention relates to gene regulation in ageing, and age-related diseases such as Alzheimer's disease. All multicellular organisms undergo the progressive and irreversible physiological decline that characterizes ageing. Postulated causes of ageing include cumulative damage to DNA leading to genomic instability, epigenetic alterations that lead to altered gene expression patterns, telomere shortening in replicative cells, oxidative damage to critical macromolecules and nonenzymatic glycation of long-lived proteins (Jazwinski (1996) Science 273:54; Martin, et al. (1996) Nature Gen. 13:25; Johnson, et al. (1996) Cell 96:291; Beckman, et al. (1998) Physiol. Revs. 78:547).

Genetic manipulation of the aging process has been achieved in Drosophila, through the over-expression of catalase and Cu/Zn superoxide dismutase (Orr, et al. (1994) Science, 263:1128, Parkes, et al. (1998), Nat. Genet. 19:171), in the nematode C. elegans, through alterations in the insulin receptor signaling pathway (Ogg, et al. (1997) Nature 389:994; Paradis et al. (1998) Genes Dev. 12:2488-2498; Tissenbaum, et al. (1998) Genetics 148:703), and through the selection of stress-resistant mutants in either organism (Johnson (1990) Science 249:908; Murakami, et al. (1996) Genetics 143:1207; Lin, et al. (1998) Science 282:943). In mammals, there has been limited success in the identification of genes that control aging rates.

Age-related changes in central nervous system function have often been associated with the loss of cells. A marked reduction in certain neurotransmitter receptor systems has been associated with increased oxidation of proteins. It has also been hypothesized that aging is associated with multiple minor periods of ischemia (multi-infarct conditions or transient ischemia attacks) which, over a period of time, may give rise to the production of oxidized protein. Other age-related dysfunctions and neurodegenerative diseases in the central nervous system have been associated with the build-up of oxidized proteins and oxidized macromolecules within neurons throughout the central nervous system.

There has been limited success in identifying genes that control ageing in mammals. Thus, a need exists to identify and characterize such genes and their expressed proteins, and to use this information to screen for individuals who have a propensity for developing an age-related disease.

### SUMMARY OF THE INVENTION

The invention is based on the discovery that a certain number of mammalian genes show either an increased, or decreased expression with age which may be important and age-related diseases such as Alzheimer's disease, or diseases arising due to oxidative stress. This provides a “genetic signature” of the ageing process. The invention, in particular relates to methods for screening a subject for a propensity for developing an age-related disease by examining the expression profile of at least one gene associated with ageing.

Accordingly, in one aspect, a method of assessing oxidative stress in a subject, by obtaining a sample of nucleic acid from the subject, measuring the level of expression associated with at least one metal ion homeostasis gene in the sample, and comparing the measured level with at least one reference value. A high level of expression indicates a heightened level of oxidative stress in the individual.

Examples of metal ion homeostasis gene include, but are not limited to a metal lothionein 1G gene, a metallothionein 1B gene, a metallothionein 2A gene, a haem binding protein 2 gene, and a haemoglobin gene.

In another aspect, the invention pertains to a method of assessing an age-related condition in a subject, by obtaining a sample of nucleic acid from the subject, measuring the level of expression associated with at least one metal ion homeostasis gene and at least one hormone gene, and comparing the measured level with at least one reference value. High levels of expression of the metal ion homeostasis gene and the hormone gene indicate an age-related condition in the subject.

The metal ion homeostasis gene can be any one of the metal ion homeostasis genes listed above. Examples of a hormone gene showing a high level of expression include, but are not limited to, an insulin receptor gene, an orexin receptor gene, a vascular endothelial growth factor gene, and a secreted frizzled related protein-1 gene. In a preferred embodiment, the hormone gene showing a high level of expression is an orexin receptor gene. In another preferred embodiment, the hormone gene showing a high level of expression is a secreted frizzled related protein-1 gene. The invention further comprises measuring the expression level of a hormone gene that shows a low level of expression compared to at least one reference value. Examples of a hormone gene showing a low level of expression include, but are not limited to, a proenkephalin gene, a somatostatin gene, and a cholecystokinin B receptor gene. In a preferred embodiment, the hormone gene showing a low level of expression is a proenkephalin gene.

The method can further comprise measuring the level of expression of at least one calcium homeostasis gene selected from the group consisting of calmodulin 1, CaM kinase II, and calbindin 1, where a low level of expression of the calcium hormone gene indicates an age-related condition in the subject. In a preferred embodiment, the calcium homeostasis gene is a calmodulin 1 gene.

The sample to be examined can be isolated from a tissue, such as the olfactory neuroepithelium, skin, brain, spinal cord, heart, liver, and the like. The sample may also be isolated from a body fluid, such as blood, serum or cerebrospinal fluid.

Any number of sequences, and any combination of genes can be examined. For example, the expression patterns of at least two sequences, at least three sequences, at least four sequences, at least five sequences, at least six sequences, at least seven sequences, at least eight sequences, at least nine sequences, at least ten sequences, can be determined, or at least 20 sequences, or at least 30 sequences.

### BRIEF DESCRIPTION OF DRAWINGS

FIG. 1 is a bar graph showing the relative changes in gene ontology categories in the aged cortex;

FIG. 2 shows the effects of limited oxidative stress and hOGG1 overexpression on cell survival;

FIG. 3A is a bar graph showing mRNA levels of selected synaptic, calcium homeostasis and transport-related genes in the aged frontal cortex determined by microarray analysis and quantitative RT-PCR;

FIG. 3B is an immunoblot showing age-related protein levels from five young and four aged frontal cortical samples;

FIG. 4A is a bar graph of mitochondrial F1 ATP synthase α mRNA;

FIG. 4B is a western blot of mitochondrial F1 ATP synthase α protein, which were reduced by about 2.5-fold in SH-SY5Y cells by siRNA transfection;

FIG. 4C is a knockdown of ATP synthase α induces promoter DNA damage in age-down regulated genes;

FIG. 4D shows promoter DNA damage determined in genomic DNA;

FIG. 5A is a graph of genomic DNA from fetal cortex showing that the fetal cortex does not exhibit significant DNA damage;

FIG. 5B are real-time fluorescence PCR curves from 26- and 77-year-old frontal cortical samples showing that ageing increases oxidative DNA damage to the mitochondrial ATP synthase α (ATP5A1α) promoter;

FIG. 5C are graphs showing a time course of DNA damage in the ageing frontal cortex. DNA damage was assayed in the promoters of age-downregulated genes;

FIG. 5D shows DNA damage to promoters of genes that are stably expressed, downregulated or upregulated in the aged cortex;

FIG. 5E is a bar graph showing oxidative damage to gene promoters in the aged cortex;

FIG. 5F shows a photograph of chromatin immunoprecipitation of the calmodulin 1 promoter with a monoclonal antibody to 8-oxoguanine in aged (≧73-year-old) and young (<40-year-old) cortical samples. Input DNA and non-specific IgG (IgG) controls are shown;

FIG. 6A is a graph depicting that promoters of age-downregulated genes show increased vulnerability to oxidative DNA damage;

FIG. 6B is a graph depicting mRNA expression of the tau gene determined in cells that overexpress the DNA repair enzyme human OGG1 (SY5Y/hOGG1) or the empty pcDNA3 vector (SY5Y);

FIG. 6C shows the mRNA levels of age-downregulated genes are selectively reduced by oxidative stress and restored by human OGG1;

FIG. 6D, shows the increased vulnerability to oxidative DNA damage in promoters of age-downregulated genes;

FIG. 6E show the reduced transcriptional activity of promoters of age-downregulated genes following oxidative DNA damage;

FIG. 6F shows that ultraviolet damage does not discriminate between promoters of age-stable and age-downregulated genes; and

FIG. 6G is a graph showing DNA damage and repair of the β-tubulin and calmodulin 1 (CaM1) promoters.

### DETAILED DESCRIPTION

The practice of the present invention employs conventional methods of virology, microbiology, molecular biology and recombinant DNA techniques within the skill of the art. Such techniques are explained fully in the literature. (See, e.g., Sambrook, et al. Molecular Cloning: A Laboratory Manual (Current Edition); DNA Cloning: A Practical Approach, Vol. I & II (D. Glover, ed.); Oligonucleotide Synthesis (N. Gait, ed, Current Edition); Nucleic Acid Hybridization (B. Hames & S. Higgins, eds., Current Edition); Transcription and Translation (B. Hames & S. Higgins, eds., Current Edition); CRC Handbook of Parvoviruses, Vol. I & II (P. Tijessen, ed.); Fundamental Virology, 2nd Edition, Vol. I & II (B. N. Fields and D. M Knipe, eds.)).

So that the invention is more clearly understood, the following terms are defined:

The term “age-related condition” or “age-related disease” or “age-related disorder” are used interchangeably herein is intended to encompass diseases and conditions associated with ageing as well as any heightened likelihood of future manifestation of such diseases or conditions, including the vulnerability or susceptibility to such diseases and conditions. These terms are also intended to include an impairment of normal cellular, physiological and mental function that occurs during the ageing process.

The term “age-related gene” as used herein refers to a nucleic acid (e.g. RNA, DNA), that is involved in aging.

The term “oxidative stress” as used herein refers to the level of damage produced by oxygen free radicals in a subject. The level of damage depends on how fast reactive oxygen species are created and then inactivated by antioxidants.

The term “free radical” as used herein refers to molecules containing at least one unpaired electron. Most molecules contain even numbers of electrons, and their covalent bonds normally consist of shared electron pairs. Cleavage of such bonds produces two separate free radicals, each with an unpaired electron (in addition to any paired electrons). They may be electrically charged or neutral and are highly reactive and usually short-lived. They combine with one another or with atoms that have unpaired electrons. In reactions with intact molecules, they abstract a part to complete their own electronic structure, generating new radicals, which go on to react with other molecules. Such chain reactions are particularly important in decomposition of substances at high temperatures and in polymerization. In the body, oxidized (see oxidation-reduction) free radicals can damage tissues. Antioxidant nutrients (e.g., vitamins C and E, selenium, polyphenols) may reduce these effects. Heat, ultraviolet light, and ionizing radiation all generate free radicals. Free radicals are generated as a secondary effect of oxidative metabolism. An excess of free radicals can overwhelm the natural protective enzymes such as superoxide dismutase, catalase, and peroxidase. Free radicals such as hydrogen peroxide (H₂O₂), hydroxyl radical (HO.), singlet oxygen (¹O₂), superoxide anion radical (O.₂⁻), nitric oxide radical (NO.), peroxyl radical (ROO.), peroxynitrite (ONOO⁻) can be in either the lipid or aqueous compartments.

The term “neurodegenerative disorder” or “neurodegenerative disease” are used interchangeably herein and refer to an impairment or absence of a normal neurological function, or presence of an abnormal neurological function in a subject, or group of subjects. For example, neurological disorders can be the result of disease, injury, and/or aging. As used herein, neurodegenerative disorder also includes neurodegeneration which causes morphological and/or functional abnormality of a neural cell or a population of neural cells. Non-limiting examples of morphological and functional abnormalities include physical deterioration and/or death of neural cells, abnormal growth patterns of neural cells, abnormalities in the physical connection between neural cells, under- or over production of a substance or substances, e.g., a neurotransmitter, by neural cells, failure of neural cells to produce a substance or substances which it normally produces, production of substances, e.g., neurotransmitters, and/or transmission of electrical impulses in abnormal patterns or at abnormal times. In a preferred example, the neurodegenerative disease is associated with the build-up of a beta amyloid protein, such as Alzheimer's disease that has a build up of plaques. Neurodegeneration can occur in any area of the brain of a subject and is seen with many disorders including, for example, Alzheimer's disease, Huntington's disease, Parkinson's disease, senile dementia, akathesia, amnesia, bipolar disorder, catatonia, cerebrovascular disease Creutzfeldt-Jakob disease, dementia, depression, tardive dyskinesia, dystonias, epilepsy, multiple sclerosis, neuralgias, neurofibromatosis, neuropathies, and schizophrenia.

The terms “modulate” or “modulating” or “modulated” are used interchangeable herein and refer to a change in the expression of at least one gene, or a plurality of genes associated with ageing or an age-related disease, i.e., an increase or decrease in expression or activity, such that the modulation produces a therapeutic effect in a subject, or group of subjects. A therapeutic effect is one that results in an amelioration in the symptoms, or progression of the age-related disease. The quantitative PCR assay can be used to measure downregulation or upregulation of a gene, or as plurality of genes associated with an age-related disease. Alternatively, the expression profile can be determined by microarray analysis, as described in the Examples. The gene(s) can be modulated by administering or delivering a therapeutic agent that reduces DNA damage, such as a DNA repair enzyme.

A suitable DNA repair enzyme can be one that increases gene expression of genes that show a decreased expression in age-related diseases, such as mitochondrial genes and synaptic transmission genes. The increase in gene expression in the presence of the therapeutic agent can be by about 1-fold, preferably by about 2-fold, 3-fold, 4-fold, 5-fold to about 10-fold, to about 20,-fold, 30-fold, 40-fold, 50-fold, 60-fold, 70-fold, 80-fold, 90-fold, 100-fold compared with a control.

The term “DNA microarray” or “DNA chip” refers to assembling PCR products of a group of genes or all genes within a genome on a solid surface in a high density format or array. General methods for array construction and use are available (Schena Science (1995) 270: 467-70). A DNA microarray allows the analysis of gene expression patterns or profile of many genes to be performed simultaneously by hybridizing the DNA microarray comprising these genes or PCR products of these genes with cDNA probes prepared from the sample to be analyzed. DNA microarray or “chip” technology permits examination of gene expression on a genomic scale, allowing transcription levels of many genes to be measured simultaneously. Briefly, DNA microarray or chip technology comprises arraying microscopic amounts of DNA complementary to genes of interest or open reading frames on a solid surface at defined positions. This solid surface is generally a glass slide, or a membrane (such as nylon membrane). The DNA sequences may be arrayed by spotting or by photolithography. Two separate fluorescently-labeled probe mixes prepared from the two sample(s) to be compared are hybridized to the microarray and the presence and amount of the bound probes are detected by fluorescence following laser excitation using a scanning confocal microscope and quantitated using a laser scanner and appropriate array analysis software packages. Cy3 (green) and Cy5 (red) fluorescent labels are routinely used in the art, however, other similar fluorescent labels may also be employed. To obtain and quantitate a gene expression profile or pattern between the two compared samples, the ratio between the signals in the two channels (red:green) is calculated with the relative intensity of Cy5/Cy3 probes taken as a reliable measure of the relative abundance of specific mRNAs in each sample. Materials for the construction of DNA microarrays are commercially available (Affymetrix (Santa Clara Calif.) Sigma Chemical Company (St. Louis, Mo.) Genosys (The Woodlands, Tex.) Clontech (Palo Alto Calif.) and Corning (Corning N.Y.). In addition, custom DNA microarrays can be prepared by commercial vendors such as Affymetrix, Clontech, and Corning.

The basis of gene expression profiling via microarray technology relies on comparing an organism under a variety of conditions that result in alteration of the genes expressed. A single population of cells may be exposed to a variety of stresses that will result in the alteration of gene expression. Alternatively, the cellular environment may be kept constant and the genotype may be altered. Typical stresses that result in an alteration in gene expression profile will include, but is not limited to conditions altering the growth of a cell or strain, exposure to mutagens, antibiotics, UV light, gamma-rays, x-rays, phage, macrophages, organic chemicals, inorganic chemicals, environmental pollutants, heavy metals, changes in temperature, changes in pH, conditions producing oxidative damage, DNA damage, anaerobiosis, depletion or addition of nutrients, addition of a growth inhibitor, and desiccation.

The term “metal ion homeostasis gene” as used herein refers to gene that maintains a normal balance of metal ions in a subject. The term also includes a gene that encodes a protein which sequesters excess ions from a cell (e.g., a neural cell), by binding to excess ions and forming a protein-ion complex. Excess amounts of these ions are toxic and cause harmful effects in the cell. This protein-ion complex can be stored in a cell or discarded from the body. Examples of ions that can be sequestered include, but are not limited to, iron, copper, zinc, and the like. In a preferred embodiment, the metal ion being sequestered is iron. Examples of metal ion homeostasis genes include, but are not limited to, a metallothionein 1G gene, a metallothionein 1B gene, a metallothionein 2A gene, a haem binding protein 2 gene, and a haemoglobin gene. The upregulation of at least one metal ion homeostasis gene compared with at least one reference value, can be indicative of ageing or an age-related disorder.

The term “hormone gene” as used herein refers to gene that is involved in hormone regulation and balance in a subject. In one embodiment, the hormone gene is one that has a higher level of expression during ageing compared with at least one reference value. Examples of a hormone gene that have a higher level of expression, include, but are not limited to, an insulin receptor gene, an orexin receptor gene, a vascular endothelial growth factor gene, and a secreted frizzled related protein-1 gene. In one preferred embodiment, the hormone gene that has a higher level of expression is the orexin receptor gene. In another preferred embodiment, the hormone gene that has a higher level of expression is the secreted frizzled related protein-1 gene. In another embodiment, the hormone gene is one that has a lower level of expression during ageing compared with at least one reference value. Examples of a hormone gene that have a lower level of expression, include, but are not limited to, a proenkephalin gene, a somatostatin gene, and a cholecystokinin B receptor gene. In a preferred embodiment, the hormone gene that has a lower level of expression is the proenkephalin gene. It will be appreciated that the invention also relates to measuring at least one hormone gene that is upregulated, at least one hormone gene that is downregulated, or a combination thereof.

The term “calcium homeostasis gene” as used herein refers to gene involved in maintaining a calcium balance in a subject. The term also refers to a gene that is involved in calcium signaling pathways, such as the calcium-calmodulin pathways. Examples of calcium homeostasis genes include, but are not limited to, calmodulin 1, CaM kinase II, and calbindin 1. In a preferred embodiment, the homeostasis gene is calmodulin 1. The downregulation of at least one calcium homeostasis gene compared with at least one reference value, can be indicative of ageing or an age-related disorder.

The phrase “altered gene expression” refers to the change in the level of a transcription or translation products. If the gene is “up-regulated”, the level of transcription or translation products is increased/elevated. If the gene is “down-regulated” the level of transcription or translation products is decreased. Alterations in gene expression may arise due to a change the environment which include but are not limited to physical properties, such as radiation fluence, radiation spectrum, humidity, substratum, or temperature; nutritional properties, such as carbon source, energy source, nitrogen source, phosphorus source, sulfur source, or trace element sources; biological properties, such as presence of competitors, predators, commensals, pathogens such as phage and other viruses, the presence of toxins, or bacterocins; and chemical properties, such as presence of chelators, inhibitors, toxicants or abnormal levels of normal metabolites that arise during ageing. Preferred genes that are down-regulated include, but are not limited to, Ca²⁺ homeostasis/signalling genes (calmodulin 1, CaM kinase II, and calbindin 1 (28 kD); Synaptic transmission genes GluR1, NMDA receptor 2A, GABA A receptor, and EAAT2 (protein)), mad box transcription enhancer factor 2C (MEF2C), and certain down-regulated hormone genes (proenkephalin, somatostatin, and cholecystokinin B receptor). Preferred genes that are up-regulated include, but are not limited to, inflammation genes (TNF-α, and H factor complement-1), metal ion homeostasis genes (metallothionein 1G, metallothionein 1B, metallothionein 2A, haem binding protein 2, and haemoglobin), and certain up-regulated hormone genes (insulin receptor, orexin receptor, vascular endothelial growth factor, and secreted frizzled related protein-1).

The term “reference value” is intended to encompass any standard or normal level of expression that is useful as a benchmark against which “aletred gene expression” can be measured. One skilled in the art can select a reference value in a myriad of ways so long as statistical relevant measurements can be obtained. For example, a reference level, or reference value for expression of a particular gene can be selected as the average level exhibited by healthy young adults (e.g., aged 25 to 30 years old). Other standards or normal reference values can be chosen depending upon the particular applications.

The phrase “gene expression pattern” refers to the expression of groups of genes (e.g., RNA, DNA), as well as the proteins they encode.

The phrase “gene expression profile” refers to the expression of individual gene (e.g., RNA, DNA), as well as the protein it encodes.

The term “gene” refers to a nucleic acid fragment that expresses a specific protein, including regulatory sequences (e.g., promoter) preceding (5′ non-coding sequences) and following (3′ non-coding sequences) the coding sequence, unless mentioned otherwise.

The term “promoter” refers to a DNA sequence to which RNA polymerase can bind to initiate the transcription. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity. “promoter region” is promoter and adjacent areas whose function may be modulate promoter activity.

The term “subject” as used herein refers to any living organism capable of eliciting an immune response. The term subject includes, but is not limited to, humans, nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be covered.

### I. Age-Related Conditions

The methods and compositions of the invention can be used to identify a subject at risk of developing an age-related conditions. Examples of age-related diseases include but are not limited to, neurodegenerative disorders of the brain and nervous system such as Alzheimer's disease, Huntington's disease, Parkinson's disease, senile dementia, akathesia, amnesia, bipolar disorder, catatonia, cerebrovascular disease Creutzfeldt-Jakob disease, dementia, depression, tardive dyskinesia, dystonias, epilepsy, multiple sclerosis, neuralgias, neurofibromatosis, neuropathies, and schizophrenia.

The methods of the invention can be used by isolating a cell sample containing nucleic acid from a subject, and examining the expression profile of at least one gene involved in an age-related condition. The nucleic acid sample can be derived from a tissue such as epithelia, olfactory neuroepithelium, brain tissue, heart tissue, muscle tissue, skin, liver tissue, skeletal tissue. The sample may also be isolated from a fluid such as blood, serum, and cereobrospinal fluid. The expression profile is then compared to at least one predetermined reference value for an age-related disease, to identify the subject's predisposition for an age-related disease. Genes involved in ageing are outlined in Tables 1-2.

PCR based amplification strategy or a DNA microarray hybridization strategy can be used to quantify the mRNA and can be used to establish profiles and expression patterns, as described in the Examples section.

In one embodiment, the age related disorder involves oxidative stress. The methods of the invention can also be used for assessing and diagnosing against certain disorders that arise from oxidative stress and the presence of excess free radicals in a subject. Free radicals are molecules containing at least one unpaired electron. They may be electrically charged or neutral and are highly reactive and usually short-lived. They combine with one another or with atoms that have unpaired electrons. In reactions with intact molecules, they abstract a part to complete their own electronic structure, generating new radicals, which go on to react with other molecules. In the body, oxidized (see oxidation-reduction) free radicals can damage tissues. Ageing, heat, ultraviolet light, and ionizing radiation all generate free radicals. An excess of free radicals can overwhelm the natural protective enzymes such as superoxide dismutase, catalase, and peroxidase. Free radicals such as hydrogen peroxide (H₂O₂), hydroxyl radical (HO.), singlet oxygen (¹O₂), superoxide anion radical (O.₂⁻), nitric oxide radical (NO.), peroxyl radical (ROO.), peroxynitrite (ONOO⁻) can be in either the lipid or compartments.

Oxidative stress can give rise to a number of pathological conditions of in a subject that results at least in part from the production of or exposure to free radicals, for example, oxyradicals, or other reactive oxygen species in vivo. Examples of free radical disorders include, but are not limited to, cataract formation, age-related macular degeneration, Alzheimer's disease, uveitis, emphysema, gastric ulcers, oxygen toxicity, neoplasia, and undesired cell apoptosis. Such diseases can include “apoptosis-related ROS” which refers to reactive oxygen species (e.g., O₂⁻) which damage critical cellular components (e.g., lipid peroxidation) in cells stimulated to undergo apoptosis, such apoptosis-related ROS may be formed in a cell in response to an apoptotic stimulus and/or produced by non-respiratory electron transport chains (i.e., other than ROS produced by oxidative phosphorylation).

In another embodiment, the age-related disease is a neurodegenerative disease associated with the central nervous system (i.e., the brain, spinal cord and CSF). Some examples of neurodegenerative disorders are as follows:

### (i) Alzheimer's Disease

Alzheimer's Disease (AD) is the most common neurodegenerative disorder of aging, and is characterized by progressive dementia and personality dysfunction. The abnormal accumulation of amyloid plaques in the vicinity of degenerating neurons and reactive astrocytes is a pathological characteristic of AD. The disease manifests itself by the presence of abnormal extracellular protein deposits in brain tissue, known as “amyloid plaques,” and tangled bundles of fibers accumulated within the neurons, known as “neurofibrillary tangles,” and by the loss of neuronal cells. The areas of the brain affected by Alzheimer's disease can vary, but the areas most commonly affected include the association cortical and limbic regions. Symptoms of Alzheimer's disease include memory loss, deterioration of language skills, impaired visuospatial skills, and impaired judgment, yet those suffering from Alzheimer's retain motor function. Patients with Alzheimer's disease exhibit reduced levels of neurotransmitter peptides: Beal et al., (1985) Science 229:289-291; Davis et al., (1980) Nature 288:279-280 and Rossor et al., (1980) Neurosci. Ltrs. 20:373-377; Whitehouse, et al. (1981) Anal. Neurol. 10:122-126 (acetyl choline); Quigley et al., (1986) Neurosci 17:70a and Quigley et al. (1991) Neurosci 41:41-60; Adolfsson et al. (1978) In “Alzheimer's Disease, Senile Dementia and Related Disorders (Aging)” Ed. Katzman et al., Vol. 7, pp. 441-451 New York, Rauer.

Alzheimer's disease is characterized by two hallmark pathological features that involve protein misfolding: Neurofibrillary tangles (NFTs) formed by paired helical filaments (PHFs) from abnormally modified Tau protein and senile plaques composed of beta-amyloid (A [symbol] ) (See Price et al. (1998) Annu Rev Neurosci 21: 479-505). Dementia and neuronal loss in Alzheimer's disease correlate significantly with levels of Tau pathology and resulting NFTs. Evidence for altered/reduced proteasomal activity in Alzheimer's disease has been found that may result from the defective ubiquination and/or breakdown of misfolded proteins such as PHF-Tau and beta amyloid by the 20S proteasome (Keck et al. (2003) J Neurochem 85:115-22; Keller et al. (2000) J Neurochem 75: 436-9; and Lopez et al., (2003) Exp Neurol 180: 131-43). Additionally, a mutant form of ubiquitin (Ub+1), generated by molecular misreading, was observed in the brains of Alzheimer's disease patients including those with the non-familial Alzheimer's disease (van Leeuwen, et al. (1998) Science 279: 242-7; and Lam, et al., (2000) Proc Natl Acad Sci USA 97: 9902-6). Ub+1 capped polyUb chain was also able to inhibit proteasomal activity in vitro and may induce accumulation of misfolded proteins and contribute to both Aβ and Tau pathology in Alzheimer's disease (Lam, et al., (2000) Supra).

A suitable animal model for Alzheimer's disease that mimics the pathology of the disease in humans can be one in which a selective lesion is placed in a subcortical nucleus (nucleus basalis of Meynert) with a resultant cortical cholinergic deficiency, similar in magnitude to that seen in early to moderate stage Alzheimer's disease. Numerous behavioral deficits, including the inability to learn and retain new information, are characteristic of this lesion. Pharmacological agents that can normalize these abnormalities would have a reasonable expectation of efficacy in Alzheimer's disease (See e.g., Haroutunian, et al. (1985) Life Sciences, 37:945-952).

In addition to in vivo models, a number of in vitro cell lines can also be used to examine the effects of pharmacological agents on Alzheimer's disease such as apolipoprotein E uptake and low-density lipoprotein receptor-related protein expression by the NTera2/D1 cell line, a cell culture model for late-onset Alzheimer's disease (See e.g., Williams et al. (1997) Neurobiol. of Disease, 4:58-67). Alternatively, human melanocytes can be used as a model system for studies of Alzheimer's disease (See e.g., Yaar et al. (1997) Arch. Dermatol. 133:1287-291).

### (ii) Parkinson's Disease

Parkinson's disease is a motor system disorder caused by the loss of nerve cells, or neurons, found in the substantia nigra region of the mid-brain. These neurons produce dopamine, a chemical messenger molecule that is found in the brain and helps control or direct muscle activity. Dopamine is used by the cells of the substantia nigra as a neurotransmitter to signal other nerve cells. Parkinson's disease occurs when these neurons die or become impaired, thereby decreasing dopamine levels within the brain. Loss of dopamine causes the neurons to fire uncontrollably, which leaves patients unable to direct or control their bodily movement in a normal manner. The four main symptoms of Parkinson's disease are trembling in the hands, arms, legs, jaw and face; stiffness of the limbs and/or trunk; a slowness of movement, referred to as bradykinesia; and impaired balance and/or coordination. Parkinson's disease is both chronic, i.e., it persists over a long period of time, and progressive, i.e., the symptoms grow worse over time.

Animal models of Parkinson's disease are well established, such as the primate model of Parkinson's Disease described by Zamir et al. (1984) Brain Res. 322, 356-60. Neurodegenerative disease-causing substance can be used to cause a neurodegenerative disease in a mammal. Examples of such substances include N-methyl-4-phenyl-1,2,3,6-tetrahydropyridine (MPTP), 1-methyl-4-henylpyridine (MPP⁺) and manganese dust for Parkinson's disease; quinolinic acid for Huntington's chorea; and beta-N-methylamino-L-alanine for amyotrophic lateral sclerosis, Parkinson's disease and Alzheimer's disease. Due to their mimicry of effects of Parkinson's disease, treatment of animals with methamphetamine or MPTP has been used to generate models for Parkinson's disease. The end result of MPTP administration is the destruction of the striatum in the brain, an area in the neocortex limbic system in the subcortical area in the center of the brain, an area compromised in Parkinson's disease. The neurotransmitter dopamine is concentrated in the striatum Parkinson's disease is characterized by lesions in that area of the brain and by depleted dopamine levels. In some species (primates) the striatal degeneration has been reported to be accompanied by behavioral symptoms that mimic Parkinson's symptoms in humans (See e.g., Markey, et al. (1986) Medicinal Research Reviews 6:389-429).

(iii) Huntington's Disease

Huntington's disease is a hereditary disorder caused by the degeneration of neurons in certain areas of the brain. This degeneration is genetically programmed to occur in certain areas of the brain, including the cells of the basal ganglia, the structures that are responsible for coordinating movement. Within the basal ganglia, Huntington's disease specifically targets nerve cells in the striatum, as well as cells of the cortex, or outer surface of the brain, which control thought, perception and memory. Neuron degeneration due to Huntington's disease can result in uncontrolled movements, loss of intellectual capacity and faculties, and emotional disturbance, such as, for example, mood swings or uncharacteristic irritability or depression.

Neuron degeneration due to Huntington's disease is genetically programmed to occur in certain areas of the brain. Studies have shown that Huntington's disease is caused by a genetic defect on chromosome 4, and in particular, people with Huntington's disease have an abnormal repetition of the genetic sequence CAG in the Huntington's disease gene, which has been termed IT15. The IT15 gene is located on the short arm of chromosome 4 and encodes a protein called huntingtin. Exon I of the IT15 gene contains a polymorphic stretch of consecutive glutamine residues, known as the polyglutamine tract (Rubinsztein, (2002) TRENDS in Genetics, 18: 202-9). Asymptomatic individuals typically contain fewer than 35 CAG repeats in the polyglutamine tract. Murine models for HD include that described by Hayden et al. in U.S. Pat. No. 5,849,995, as well as in vitro systems as described in U.S. Pat. No. 5,834,183 to Orr et al.

The skilled artisan will appreciate that the methods of the invention can be readily applied to any number of age-related diseases in which at least one age-related gene is altered during the ageing progress.

### III. Pathways

Genes identified as being up-regulated or down-regulated are involved in a variety signaling pathways. Accordingly, modulation of a pathway by altering the expression of a gene involved in the pathway may help to ameliorate age-related disorders. Examples of preferred pathways that may be involved are as follows:

### (i) Wnt Signaling Pathway

The Wnt signaling pathway is evolutionary conserved and controls many events during the embryogenesis. At the cellular level this pathway regulates morphology, proliferation, motility and cell fate. Also during tumorigenesis the Wnt signaling pathway has a central role and inappropriate activation of this pathway are observed in several human cancers (Spink et al. (2000) EMBO J. 19:2270-2279).

In the presence of a Wnt ligand, the Wnt ligand binds a frizzled (Fz)/low density lipoprotein receptor related protein (LRP) complex, activating the cytoplasmic protein dishevelled (Dsh in Drosophila and Dvl in vertebrates). Precisely how Dsh/Dvl is activated is not fully understood, but phosphorylation by casein kinase 1 (CK1) and casein kinase 2 (CK2) have been suggested to be partly responsible (Willert (1997) EMBO J. 16:3089-3096); Sakanaka, et al. (1999) Proc. Natl. Acad. Sci. U.S.A 96:12548-12552; and Amit, et al. (2002) Genes Dev. 16:1066-1076).

Dsh/Dvl then inhibits the activity of the multiprotein complex (β-catenin-Axin-adenomatous polyposis coli (APC)-glycogen synthase kinase (GSK)-3β), which targets β-catenin by phosphorylation for degradation by the proteasome. Dsh/Dvl is suggested to bind CK1 and thereby inhibiting priming of β-catenin and indirectly preventing GSK-3β phosphorylation of β-catenin. Upon Wnt stimulation, Dvl has also been shown to recruit GSK-3 binding protein (GBP) to the multiprotein complex. GBP might titrate GSK-3β from Axin and in this way inhibits phosphorylation of β-catenin. Stabilized β-catenin can then translocate into the nucleus and bind to members of the T-cell factor (Tcf)/Lymphoid enhancing factor (Lef) family of DNA binding proteins leading to transcription of Wnt target genes.

In the absence of a Wnt ligand, Axin recruits CK1 to the multiprotein complex causing priming of β-catenin and initiation of the β-catenin phosphorylation cascade performed by GSK-3β. Phosphorylated β-catenin is then recognized by β-transducin repeat-containing protein (β-TrCP) and degraded by the proteaosome, reducing the level of cytosolic β-catenin.

At least three classes of Wnt antagonists are reported in Xenopus, all with human homologues. The first class, secreted frizzled-related proteins (sFRPs), are also called secreted apoptosis-related proteins (SARPs) due to their effect on cell sensitivity to proapoptotic stimuli (Melkonyan et al. (1997) Proc. Natl. Acad. Sci. USA 94:13636-13641). They contain a cysteine-rich domain with similarity to the ligand-binding domain of the Fz transmembrane protein family, but lack the 7-transmembrane part that anchors Fz proteins to the plasma membrane (Rattner (1997) Proc. Natl. Acad. Sci. U.S.A 94:2859-2863). The sFRPs thus compete with the Fz proteins for binding to secreted Wnt ligands and antagonize the Wnt function. However, a contradictory effect of the sFRPs has been described, in which the sFRPs enhance the Wnt activity by facilitating the presentation of the ligand to the Fz receptors (Uthoff et al. (2001) Mol. Carcinog. 31:56-62). Three human homologues are identified, SARP1-3, but they show distinct expression pattern (Melkonyan et al., Supra).

### (ii) Ion Homeostasis and Sequestering

Intracellular levels of essential ions such as calcium and transistion metals such as zinc are normally maintained at low levels because pronounced elevations in may be neurotoxic. Calcium and zinc readily enter neurons via glutamate receptor and voltage gated Ca²⁺ channels. Elevations of intracellular metal ions may contribute to glutamate excitotoxicity and play a role in Alzheimer's pathology. Ion transport can be examined by direct measurement of the ion transport function in plasma membrane vesicles purified from rat brain and in neurons in primary cell culture. Imaging studies using fluorescent dyes can be used to measure changes in intracellular ion and pH.

Iron is another essential metal ion. Due to its unique chemical properties, iron plays a central role in biology. Although iron is vital for life, it is highly reactive and so can be toxic when in excess. Evolution has thus developed mechanisms to regulate the amount of iron in the cells of the body. Painstaking studies of iron balance in humans 65 years ago showed that virtually no iron is excreted and that stable iron levels are maintained by modulating absorption of iron from the gut (McCance, et al. (1937) Lancet 2:680. Iron homeostasis is complex, as there are many different proteins that respond not only to the total body burden of iron, but also to stimuli such as hypoxia, anemia, and inflammation.

There are two very different aspects to iron homeostasis. First, iron modulates the synthesis of a variety of proteins involved in iron metabolism, including the iron storage protein ferritin, the iron transporter transferrin, and the transferrin receptor. Second, another group of proteins regulates the transport of iron into and out of cells. In response to iron deficiency, hypoxia, or anemia, more iron is transported out of the gut lumen into intestinal epithelial cells, and then from the intestinal epithelia and liver macrophages (in the form of iron recycled from hemoglobin) into the blood. Inflammation and iron overload have the opposite effect, decreasing the amount of iron absorbed from the gut and released into the blood. The regulation of iron metabolism proteins by iron and the control of iron transport are undoubtedly connected.

It was established that RNA motifs called iron responsive elements (IRE) in numerous transcripts of genes involved in iron metabolism and homeostasis (Rouault (2002) Blood Cells Mol. Dis. 29, 309; Rouault, (1997) Curr. Top. Cell Regul. 35, 1; and Hentze (1996) Proc. Natl. Acad. Sci. U.S.A. 93: 8175. These motifs are bound by iron regulatory proteins 1 and 2 (IRP1 and IRP2) depending on cellular iron levels. When these proteins bind to IRE motifs in the 5′-untranslated region of, for example, the ferritin mRNA transcript, translation of the transcript is blocked and synthesis of ferritin is halted. In contrast, when IRP1 and IRP2 bind to the IRE in the 3′-untranslated region of, for example, the transferrin receptor transcript, the transcript is stabilized, translation proceeds, and the transferrin receptor is synthesized.

Evolution has provided two IRPs, both of which bind to IREs but sense iron in very different. IRP1 is a bifunctional cytosolic protein that contains an iron-sulfur cluster. In the presence of iron, IRP1 acts as an aconitase (interconverting citrate and isocitrate), but in the absence of iron, IRP1 binds to the IREs of various iron homeostasis transcripts with high affinity. By contrast, IRP2 undergoes iron-dependent degradation in iron-replete cells and therefore is not available to bind to the IREs. But things are a little more complicated than this. IRP2 is also sensitive to degradation in the presence of nitric oxide (NO), whereas IRP1 is activated by NO (Meyron-Holtz et al. (2004) Science 306, 2087). It had been presumed that IRP1 is the principal iron sensor and a major player in iron homeostasis, yet mice deficient in IRP1 appear normal. In contrast, mice deficient in IRP2 show pronounced misregulation of iron metabolism and nerve damage.

(iii) Creb Pathway

Memory storage includes a short-term phase (STM) which requires the phosphorylation of pre-existing proteins, and a long-term phase (LTM) which needs the novel synthesis of RNA and proteins. Cyclic AMP and a specific transcription factor (cAMP response element binding protein or CREB) play a central role in the formation of LTM. Following its phosphorylation by protein kinase A, CREB binds to the enhancer element CRE which is located in the upstream region of cAMP-responsive genes, thus triggering transcription. Some of the newly-synthesized proteins are additional transcription factors that ultimately give rise to the activation of late response genes, whose products are responsible for the modification of synaptic efficacy leading to LTM.

### (iv) Calcineurin Pathway

Calcineurin signaling has been implicated in a broad spectrum of developmental processes in a variety of organ systems. Calcineurin is a calmodulin-dependent, calcium-activated protein phosphatase composed of catalytic and regulatory subunits. The serine/threonine-specific phosphatase functions within a signal transduction pathway that regulates gene expression and biological responses in many developmentally important cell types. Calcineurin signaling was first defined in T lymphocytes as a regulator of nuclear factor of activated T cells (NFAT) transcription factor nuclear translocation and activation. Recent studies have demonstrated the vital nature of calcium/calcineurin/NFAT signaling in cardiovascular and skeletal muscle development in vertebrates. Inhibition, mutation, or forced expression of calcineurin pathway genes result in defects or alterations in cardiomyocyte maturation, heart valve formation, vascular development, skeletal muscle differentiation and fiber-type switching, and cardiac and skeletal muscle hypertrophy.

One or more of these pathways may be involved in the genes that are up-regulated or the genes that are down-regulated. For example, both the calcineurin/Creb signal transduction pathways may be involved to enhanced transcription of the proenkephalin. This can involve a Ca²⁺ influx, followed by calmodulin/calcineurin activation, then CREB activation, and proenkephalin gene transcriptional up-regulation.

### VI. DNA Repair and Therapeutics

In one aspect, the invention pertains to ameliorating an age-related disease in a subject afflicted with, or at risk of developing an age-related disease, by administering a therapeutic agent that reduces DNA damage.

The DNA in each cell of a body is constantly subjected to damage caused by both internal (e.g., reactive oxygen species) and external DNA damaging agents (e.g., sunlight, X- and gamma-rays, smoke) that cause lesions (breaks) in the DNA. Most lesions are eliminated from DNA by one of several pathways of DNA repair. When unrepaired DNA lesions are replicated, they cause mutations because of their miscoding nature. The occurrence of such mutations in critical genes, e.g., oncogenes and tumor suppressor genes, may lead to the development of cancer. Indeed, DNA repair has emerged in recent years as a critical factor in cancer pathogenesis, as a growing number of cancer predisposition syndromes have been shown to be caused by mutations in genes involved in DNA repair and the regulation of genome stability.

Cells have evolved the capacity to remove or tolerate lesions in their DNA. The most direct mechanisms for repairing DNA are those that simply reverse damage and restore DNA to its normal structure in a single step. A more complex mechanism, excision repair, involves incision of the DNA at the lesion site, removal of the damaged or inappropriate base(s), and resynthesis of DNA using the undamaged complementary strand as a template. This system of repair can further be categorized into base and nucleotide excision repair.

DNA base excision repair (BER) may work through two alternative pathways. The first involves four enzymes in mammalian cells: DNA glycosylases, such as methylpurine-DNA glycosylase (MPG), apurinic/apyrimidinic (AP) endonucleases (APE or APN-1), DNA beta-polymerase (β-Pol) and DNA ligase. DNA glycosylases are enzymes that hydrolyze the N-glycosidic bond between the damaged base and the deoxyribose moiety, creating an AP site on the DNA backbone. AP sites, whether produced by glycosylases or directly by DNA damaging agents (bleomycin) are acted upon by AP endonucleases, which can make an incision either 3′ to the AP site (AP lyase) or 5′ to the AP site (hydrolytic). The resulting gap in the phosphodiester backbone is filled in by DNA β-Pol and the ends are ligated by DNA ligase I. Examples of combined glycosylase/AP lyases include the E. coli formamidopyrimidine glycosylase (fpg), yeast and human OGG1, and Drosophila S3. The fpg glycosylase/AP lyase recognizes and initiates repair of ring-opened bases such as formamidopyrimidine-Guanine (FaPy-Gua) and methylated formamidopyrimidine (N7-methylformamidopyrimidine; 7-methyl-FaPy-Gua). These lesions that are produced by alkylating agents such as thiotepa and by oxidative DNA damaging.

DNA repair may be improved by therapeutic agents that reduce DNA damage such as a DNA repair enzyme. Examples of DNA repair enzymes include, but are not limited to, base excision repair enzymes such as OGG1, repair enzyme adenosine diphosphate ribosyl transferase (ADPRT), exoIII, endoIV, endoIII, fpg, dS3, β-Pol polymerase, and DNA ligase. Other therapeutic agents that reduce DNA damage by enhancing innate DNA repair in a subject, may also be used such as beta-lactam antibiotics.

For an age-related gene that shows an increased expression during ageing, a therapeutic agent is one that reduces the expression of the gene by silencing gene expression. For example, a silencing oligonucleotide complementary to a polynucleotide that is overexpressed. The silencing oligonucleotide can be an antisense sequence, for example an interfering RNA sequence. For an age-related gene that shows a decrease in expression during ageing, the therapeutic agent is one that increases the expression of the gene, for example by gene therapy methods that deliver a nucleic acid to a target region. Expression of the protein encoded by the nucleic acid may correct a disease state.

The therapeutic agent that reduces DNA damage can be delivered to the subject for overexpression by using an expression construct comprising a vector having an isolated nucleic acid encoding a DNA repair enzyme and a promoter operably linked to the isolated nucleic acid. The viral vector may be selected from the group consisting of a retroviral vector, an adenoviral vector, a herpesviral vector, adeno-associated viral vector and a cytomegaloviral vector. For example, the DNA repair enzyme, e.g., OGG1, can be engineered to be introduced into an expression vector and delivered to a host for expression. The vector can be delivered in vivo, in vitro, or ex vivo for expression. When the DNA repair enzyme is expressed in vitro, it can be purified using standard techniques for protein purification to produce an active enzyme that can be administered to a target cell. Other therapeutic agents are those that aid in genes involved in DNA repair, antioxidant defense, stress response, and inflammatory responses. Other known therapeutic agents include, but are not limited to, nutraceuticals and vitamins that influence aging favorably. These can be used in combination with the DNA repair enzyme.

The therapeutic agent may also be one that acts to prevent aggregation of a β-amyloid protein. Beta-amyloid is a well characterized protein that is the primary constituent of senile plaques and cerebrovascular deposits in Alzheimer's disease. Beta-amyloid protein is encoded as part of a message that encodes a much larger precursor (the amyloid precursor protein, APP), carboxy terminal fragments of which are neurotoxic to hippocampal neurons in culture (Yankner et al., (1989) Science 245: 417, Whitson et al., (1989) Science 243: 1488. Other homologous peptides of β-amyloid which increase the survival of young undifferentiated hippocampal neurons in cell culture are also known. (See U.S. Pat. No. 5,876,948, U.S. Pat. No. 6,440,387; U.S. Pat. No. 6,080,778; U.S. Pat. No. 5,876,948; U.S. Pat. No. 5,137,873; and U.S. patent applications 20020183379; 2002012000; and 20020081263, incorporated herein by reference).

### V. Expression Profiling

The application of gene expression profiling is particularly relevant to improving diagnosis, prognosis, and treatment of an age-related disease. In one aspect, the invention pertains to examining the expression pattern of at least one gene associated with an age-related disease. Because gene expression patterns are responsive to both intracellular and extracellular events, the present invention can provide the simultaneous monitoring of a plurality of genes on a tissue-specific or organ-specific basis that would reveal a set of genes that are altered in expression levels as a consequence of biological aging. A global analysis of gene expression patterns during aging identifies genes that are expressed differentially as a consequence of aging to provide a quantitative assessment of aging rates. Both the levels and sequences expressed in tissues from subjects with an age-related disease may be compared with the levels and sequences expressed in normal brain tissue during ageing.

### (i) Diagnostics

The cDNAs, or fragments thereof of genes associated with an age-related disease, may be used to detect and quantify altered gene expression; absence, presence, or excess expression of mRNAs; or to monitor mRNA levels during therapeutic intervention. These cDNAs can also be utilized as markers of treatment efficacy against the diseases and other brain disorders, conditions, and diseases over a period ranging from several days to months to years. The diagnostic assay may use hybridization or amplification technology to compare gene expression in a biological sample from a patient to standard samples in order to detect altered gene expression. Qualitative or quantitative methods for this comparison are well known in the art.

For example, the cDNA may be labeled by standard methods and added to a biological sample from a patient under conditions for the formation of hybridization complexes. After an incubation period, the sample is washed and the amount of label (or signal) associated with hybridization complexes, is quantified and compared with a standard value. If the amount of label in the patient sample is significantly altered in comparison to the standard value, then the presence of the associated condition, disease or disorder is indicated.

In order to provide a basis for the diagnosis of a condition, disease or disorder associated with gene expression, a normal or standard expression profile is established. This may be accomplished by combining a biological sample taken from normal subjects, either animal or human, with a probe under conditions for hybridization or amplification. Standard hybridization may be quantified by comparing the values obtained using normal subjects with values from an experiment in which a known amount of a substantially purified target sequence is used. Standard values obtained in this manner may be compared with values obtained from samples from patients who are symptomatic for a particular condition, disease, or disorder. Deviation from standard values toward those associated with a particular condition is used to diagnose that condition.

Such assays may also be used to evaluate the efficacy of a particular therapeutic treatment regimen in animal studies and in clinical trial or to monitor the treatment of an individual patient. Once the presence of a condition is established and a treatment protocol is initiated, diagnostic assays may be repeated on a regular basis to determine if the level of expression in the patient begins to approximate that which is observed in a normal subject. The results obtained from successive assays may be used to show the efficacy of treatment over a period ranging from several days to months.

### (ii) Gene Expression Profiles

A gene expression profile comprises a plurality of cDNAs and a plurality of detectable hybridization complexes, where each complex is formed by hybridization of one or more probes to one or more complementary sequences in a sample. The cDNA composition of the invention is used as elements on a microarray to analyze gene expression profiles. In one embodiment, the microarray is used to monitor the progression of disease. Researchers can assess and catalog the differences in gene expression between healthy and diseased tissues or cells. By analyzing changes in patterns of gene expression, disease can be diagnosed at earlier stages before the patient is symptomatic. The invention can be used to formulate a prognosis and to design a treatment regimen. The invention can also be used to monitor the efficacy of treatment.

For treatments with known side effects, the microarray is employed to improve the treatment regimen. A dosage is established that causes a change in genetic expression patterns indicative of successful treatment. Expression patterns associated with the onset of undesirable side effects are avoided. This approach may be more sensitive and rapid than waiting for the patient to show inadequate improvement, or to manifest side effects, before altering the course of treatment.

In another embodiment, animal models which mimic a human disease can be used to characterize expression profiles associated with a particular condition, disorder or disease or treatment of the condition, disorder or disease. Novel treatment regimens may be tested in these animal models using microarrays to establish and then follow expression profiles over time. In addition, microarrays may be used with cell cultures or tissues removed from animal models to rapidly screen large numbers of candidate drug molecules, looking for ones that produce an expression profile similar to those of known therapeutic drugs, with the expectation that molecules with the same expression profile will likely have similar therapeutic effects. Thus, the invention provides the means to rapidly determine the molecular mode of action of a drug.

### VI. Model Systems

Animal models may be used as bioassays where they exhibit a phenotypic response similar to that of humans and where exposure conditions are relevant to human exposures. Mammals are the most common models, and most infectious agent, cancer, drug, and toxicity studies are performed on rodents such as rats or mice because of low cost, availability, lifespan, reproductive potential, and abundant reference literature. Inbred and outbred rodent strains provide a convenient model for investigation of the physiological consequences of underexpression or overexpression of genes of interest and for the development of methods for diagnosis and treatment of diseases. A mammal inbred to overexpress a particular gene (for example, secreted in milk) may also serve as a convenient source of the protein expressed by that gene.

### (i) Transgenic Animal Models

Transgenic rodents that overexpress or underexpress a gene of interest may be inbred and used to model human diseases or to test therapeutic or toxic agents. (See, e.g., U.S. Pat. Nos. 5,175,383 and 5,767,337.) In some cases, the introduced gene may be activated at a specific time in a specific tissue type during fetal or postnatal development. Expression of the transgene is monitored by analysis of phenotype, of tissue-specific mRNA expression, or of serum and tissue protein levels in transgenic animals before, during, and after challenge with experimental drug therapies.

### (ii) Embryonic Stem Cells

Embryonic (ES) stem cells isolated from rodent embryos retain the potential to form embryonic tissues. When ES cells such as the mouse 129/SvJ cell line are placed in a blastocyst from the C57BL/6 mouse strain, they resume normal development and contribute to tissues of the live-born animal. ES cells are preferred for use in the creation of experimental knockout and knockin animals. The method for this process is well known in the art and the steps are: the cDNA is introduced into a vector, the vector is transformed into ES cells, transformed cells are identified and microinjected into mouse cell blastocysts, blastocysts are surgically transferred to pseudopregnant dams. The resulting chimeric progeny are genotyped and bred to produce heterozygous or homozygous strains.

(iii) Knockout Analysis

In gene knockout analysis, a region of a gene is enzymatically modified to include a non-natural intervening sequence such as the neomycin phosphotransferase gene (neo; Capecchi (1989) Science 244:1288-1292). The modified gene is transformed into cultured ES cells and integrates into the endogenous genome by homologous recombination. The inserted sequence disrupts transcription and translation of the endogenous gene.

### (iv) Knockin Analysis

ES cells can be used to create knockin humanized animals or transgenic animal models of human diseases. With knockin technology, a region of a human gene is injected into animal ES cells, and the human sequence integrates into the animal cell genome. Transgenic progeny or inbred lines are studied and treated with potential pharmaceutical agents to obtain information on the progression and treatment of the analogous human condition.

### VII. Delivery and Pharmaceutical Systems

Delivery systems include methods of in vitro, in vivo and ex vivo delivery of a vector carrying a therapeutic agent. For in vivo delivery, the vector can be administered to a subject in a pharmaceutically acceptable carrier. The term “pharmaceutically acceptable carrier”, as used herein, refers to any physiologically acceptable carrier for in vivo administration. Such carriers do not induce an immune response harmful to the individual receiving the composition. In another embodiment, the nucleic acid encoding the therapeutic agent can be delivered using a non-viral delivery system, such as colloidal dispersion systems that include, for example, macromolecule complexes, nanocapsules, microspheres, beads, and lipid-based systems including oil-in-water emulsions, micelles, mixed micelles, and liposomes, gene gun based delivery are described, for example by, Braun et al. (1999) Virology 265:46-56; Drew et al. (1999) Vaccine 18:692-702; Degano et al. (1999) Vaccine 18:623-632; and Robinson (1999) Int J Mol Med 4:549-555; Lai et al. (1998) Crit Rev Immunol 18:449-84; See e.g., Accede et al. (1991) Nature 332: 815-818; and Wolff et al. (1990) Science 247:1465-1468 Murashatsu et al., (1998) Int. J. Mol. Med. 1: 55-62; Agracetus et al. (1996) J. Biotechnol. 26: 37-42; Johnson et al. (1993) Genet. Eng. 15: 225-236).

If the therapeutic agent is incorporated into a pharmaceutical composition suitable for administration to a subject, typically, the pharmaceutical composition comprises the vector carrying the therapeutic agent and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the antibody or antibody portion.

The compositions of this invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans. In one embodiment, the mode of administration is parenteral (e.g., intravenous, subcutaneous, intraperitoneal, intramuscular). In another embodiment, the mode of administration is by intravenous infusion or injection. In another embodiment, the mode of administration is by intramuscular or subcutaneous injection.

The compositions of the invention may include a “therapeutically effective amount” or a “prophylactically effective amount” of a vector of the therapeutic agent. A “therapeutically effective amount” refers to an amount effective, at dosages and for periods of time necessary, to achieve the desired therapeutic result. A therapeutically effective amount of the therapeutic agent may vary according to factors such as the disease state, age, sex, and weight of the individual. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Variations, modifications, and other implementations of what is described herein will occur to those of ordinary skill in the art without departing from the spirit and scope of the invention as claimed. Accordingly, the invention is to be defined not by the preceding illustrative description but instead by the spirit and scope of the following claims. All articles, patents, and patent applications cited herein are incorporated by reference.

### EXAMPLES

### Example 1

### Methods and Materials

### (i) DNA Microarray Analysis

Thirty cases spanning ages of 26 to 106 were used for microarray analysis Dissections of the frontal pole were performed and tissue samples were snap frozen in liquid nitrogen. Total RNA was extracted and complementary RNA targets were prepared, labelled and hybridized with an Affymetrix Test 3 Array. Samples with acceptable RNA quality were hybridized to Affymetrix HG-U95Av2 oligonucleotide arrays representing about 12,000 probe sets. Three approaches were used to analyse the data. (Yankner, (2000) Nature 404, 125). Arrays were normalized and genes that correlated with age (Spearman rank correlation P-value <0.005) were determined and resolved by hierarchical clustering using dChip V1.3 software. (Lee (2000) Nature Genet. 25, 294-297. Correlation coefficient analysis was performed to assess the relatedness of each case to every other case using S-PLUS 2000 software (Insightful Corp.). Gene-wise standardized expression values of the genes that show Spearman rank correlation with age were used to compute Pearson correlation coefficients between two cases. The correlation coefficient matrix containing all pairwise correlation coefficients was then read into dChip for heat-map visualization. The range of observed correlation coefficients was 0.77 to 0.80, and 0.7 was used as the display range (correlation above 0.7 is pure red, below 0.7 is pure blue, and 0 is white) (Jiang, (2001) Proc. Natl Acad. Sci. USA 98, 1930-1934). Significance analysis of microarrays (SAM) software was used to compare young (≦42 years old) and aged (≧73 years old) groups to determine the list of genes with a ≧1.5-fold change and median false discovery rate (FDR)<0.01 as shown in Tables 2 and 3 (Tusher et al. (2001) Proc. Natl. Acad. Sci. USA 98, 5116-5121. Some of the DNA microarray results were validated by quantitative reverse transcription (RT)-PCR and western blot analysis.

### (ii) DNA Damage Analysis

The isolation of genomic DNA for the analysis of oxidative DNA damage was performed under conditions that prevent in vitro oxidation, including the presence of 50 μM of the free-radical spin trap phenyl-tert-butyl nitrone (PBN, Sigma), nitrogenation of all buffers, and avoidance of phenol and high temperature. Fetal brain genomic DNA (18 weeks gestation) isolated under these conditions did not show significant oxidative damage. DNA damage was assayed by cleavage of genomic DNA with FPG (New England Biolabs), which acts as an efficient N-glycosylase and AP-lyase to excise 8-oxoguanine and other damaged bases, and creates a single-strand break that prevents PCR amplification. Quantitative RT-PCR was then used to determine the content of specific intact sequences. The ratio of PCR products after FPG cleavage to those present in uncleaved DNA was used to determine the percentage of intact DNA. Incorporation of 8-oxoguanine was assayed by cleavage of genomic DNA with the 8-oxoguanine-specific N-glycosylase human OGG1 (New England Biolabs) and by chromatin immunoprecipitation with a monoclonal antibody to 8-oxoguanine.

(iii) Cell Culture

Stable cell lines were derived from human neuroblastoma SH-SY5Y cells by transfection with a pcDNA3.1 vector encoding His-tagged human nuclear human OGG1 (a gift from G. Verdine). Stable clonal cell lines were derived by selection in medium containing G418, and human OGG1 expression was confirmed by western blotting. Cells stably expressing the empty pcDNA3.1 vector were used as controls. Human cortical neuronal cultures were established as previously described by Xu (2002) Nature Med. 8, 600-606. Cells were subjected to a mild pro-oxidative stress by treatment with H₂O₂ and FeCl₂, which did not reduce cell viability during the treatment period.

### (iv) Luciferase Reporter Assays

Gene promoter sequences were identified based on published literature or predictions from the genome data base, PCR-amplified from human brain genomic DNA, and cloned in the luciferase reporter vector pGL3-basic (Promega). The host cell reactivation assay of DNA repair was performed by treating luciferase reporter plasmids with 100 μM H₂O₂ for one hour in vitro, or by exposing the DNA to ultraviolet-C light (254 nm) at 200 J/m² (Athas (1991) Cancer Res. 51, 5786-5793). Damaged or control non-damaged promoter reporter plasmids were transfected into SH-SY5Y or SH-SY5Y/human OGG1 cells together with a pRL-TK-Renilla control plasmid (Promega) using Lipofectamine 2000 (Invitrogen). Sixteen hours after transfection, cells were lysed and analysed by the Dual-Luciferase Reporter Assay (Promega). Reporter luciferase activity was normalized to renilla-luciferase activity to control for transfection efficiency. The luciferase activity of H₂O₂ or ultraviolet-damaged reporters was expressed as the percentage of the luciferase activity of the corresponding non-damaged reporters. To assess DNA damage in the promoter regions of the transfected reporters, the FPG cleavage/PCR-based assay was used with PCR primers against regions of the pGL3 plasmid that encompassed the cloned promoters. This excluded amplification of endogenous promoter sequences of the target genes.

### (v) Postmortem and Biopsy Cases

Detailed case information is provided in Table 2. The postmortem brain tissue samples used in this study were neuropathologically normal for age, and were derived from non-demented individuals. Some cases had been neuropsychologically tested as part of aging studies (77, 80, 82, 87, 88, 90 (B) and 91 years old). Tissue was procured in accordance with institutional guidelines. Human frontal cortical grey matter samples were dissected from the frontal pole (Brodmann area 10), and were snap frozen in liquid nitrogen and stored at −85° C. Some intracortical biopsy samples were also included in this study. Cluster and correlation coefficient analysis utilized 30 cases (Table 2; 26, 26B, 27, 29, 30, 36, 37, 38, 40, 42, 45, 48, 52, 53, 56, 61, 66, 70, 71, 73, 77, 80, 81, 85, 87, 90, 90B, 91, 95 and 106 years old). Group comparison (Table 1 and Table 3) utilized cases ≦42 years old (26, 26B, 27, 29, 30, 36, 37, 38, 40 and 42 years) and ≧73 years old (73, 77, 80, 81, 85, 87, 90, 90B, 91, 95, and 106 years).

### (vi) RNA Isolation and Microarray Hybridization

Dissected cortical grey matter was cut into small pieces in the frozen state and ˜70 mg was homogenized immediately in Trizol (Gibco) and RNA was isolated. RNA that was intact by electrophoresis and had an A₂₆₀/A₂₈₀ ratio ≧1.9 was used for cDNA synthesis. cDNA, cRNA synthesis, cRNA fragmentization and preparation of the hybridization cocktail were carried out according to the Affymetrix protocol. After hybridization for 16 hrs at 45° C. in the Genechip hybridization oven 640 (60 vrpm), the probe arrays were washed, stained in the GeneChip Fluidics Station 400 operated by GeneChip software following the appropriate fluidics protocols, e.g. micro1v1 for test3 chips and EukGE-WS2v4 for U95Av2 chips. The Microarray Suite Software controlled HP G2500A GeneArray Scanner was utilized to scan the surface of probe arrays and the converted digital intensity values were stored as image data files (*data) for further data analysis. All hybridization cocktails were pre-screened by test3 chips, and only those with GAPDH 3′:5′ ratios <3 were chosen for hybridization onto U95AV2 chips. Replicate or triplicate hybridizations of individual samples were performed with correlation coefficients ≧0.98.

(vii) Microarray and Statistical Data Analysis

The dChip V1.3 software was used to normalize the 30 CEL files at probe level and compute model-based expression values using the PM/MM difference model (Li, et al. (2001) Proc. Natl. Acad. Sci. U.S.A. 98, 31-36). A presence call threshold of ≧20% was required. dChip was also used for supervised correlation filtering using age information (Spearman rank correlation P-value <0.005), and to visualize the expression data by hierarchically clustering genes and samples (Eisen, et al. (1998) Proc. Natl. Acad. Sci. U.S.A. 95, 14863-14868). In the hierarchical clustering of genes, “1-Pearson's correlation of two genes across samples” was used as the distance metric between two genes, and the centroid linkage method was used to compute the distance between a gene and a gene cluster and between two gene clusters. This involves computing the standardized expression values (scaled to have mean 0 and standard deviation 1) of a gene across samples, averaging the standardized values of genes sample-wise in a gene cluster, and using this averaged expression profile as the expression vector of a gene cluster to compute distance between gene clusters. The standardized values of genes are displayed a cluster Figure according to the color scale and display range, with red color representing above-average expression levels and blue color representing below-average expression levels (data not shown).

The correlation coefficient between samples was computed using S-PLUS 2000 software (Insightful Corporation) based on the gene-wise standardized expression values of genes that show Spearman rank correlation with age. The correlation matrix was saved into text file and read into dChip for heatmap visualization.

The two-sample comparison of young cases ≦42 years old and aged cases ≧73 years old was performed using Significance Analysis of Microarrays (SAM) software with 5000 permutations and a 6-value of 1.097 to generate a list of 463 genes with fold change ≧1.5 and median false discovery rate (FDR) <0.01 (Tusher, et al. (2001) Proc Natl Acad Sci USA. 98, 5116-5121). The presence call percentage applies to all samples and thus is equivalent to applying the same presence call filter to all the permutated datasets in the SAM procedure.

Gene Ontology annotations were based on the NetAffx annotation files (Blalock (2003) Neurosci. 23, 3807-3819), which in turn were based on the LocusLink database, (Kandel (2001) Science 294, 1030-1038 and Gene Ontology database (Malinow (2002) Annu. Rev. Neurosci. 25, 103-126).

Simple linear regression models (Stata 8.1) were used to evaluate the relationship between gene expression and tissue postmortem interval (PMI). Two types of analysis were performed: (i) mRNA expression level was plotted against PMI for individual genes in each sample. Twenty age-downregulated and twenty age-upregulated genes were individually analyzed, and (ii) a cumulative measure of normalized values of all genes in the age-downregulated cluster or the age-upregulated cluster was determined in each sample and plotted against the PMI. Both types of analysis failed to demonstrate a statistically significant relationship between DNA microarray results and PMI (P-value >0.05).

(viii) Quantitative Real Time PCR/RT-PCR

Real time quantitative PCR/RT-PCR was carried out on an iCycler iQ system (BioRad) using SYBR Green one step PCR/RT-PCR kits (Qiagen). All reactions were performed in a 25 μl mixture containing 1×SYBR reaction buffer, 0.5 μM primers (forward and backward), 10 nM fluorescein calibration dye (Bio-Rad), and 10 ng genomic DNA or 1 ng total RNA for QPCR and QRT-PCR, respectively. A standard curve derived from 10-fold serial dilutions of purified PCR products of the target gene was used to determine absolute concentrations of target RNA/DNA. Primers were generally 18-25 bp long with Tms around 60° C. For RT-PCR, primers were designed to cross intron-exon boundaries, with product lengths ranging from 90 to 150 bp. 18S rRNA was used as a reference gene for the internal control. For PCR amplification of promoters, primers were designed to encompass ˜0.5 kb upstream of the transcription initiation site. Negative controls (absence of template or reverse transcriptase for RT-PCR) were used to monitor nonspecific amplification. PCR products were verified by electrophoresis. Fluorescence from incorporated SYBR Green was captured at the end of each cycle and continuously during the melting curves. The fluorescence threshold value was determined automatically by the iCycle iQ system software, and was further converted into concentration according to the standard curve. For QRT-PCR, the concentration of a given gene was normalized to the 18S rRNA internal control.

### (ix) Immunoblot Analysis

Brain tissues samples were homogenized with a glass Dounce tissue grinder (Kontes) in RIPA-DOC buffer (50 mM Tris buffer pH 7.2, 150 mM NaCl, 1% Triton-X100, 1% deoxycholate and 0.1% SDS) supplemented with protease inhibitors (Complete, Roche Molecular Biochemicals) as well as phosphotase inhibitors (50 mM NaF, 5 mM Na₂P₂O₇, 1 mM NaVO₄, 1 μM microcysteine). SDS buffer (10 mM Tris buffer, pH 7.2, 100 mM NaCl, 2 mM EDTA, 1% SDS) and incubation at 100° C. for 5 min was used to extract proteins for the analysis of tau. Protein concentrations were quantified with the DC protein assay kit (Bio-Rad) and adjusted to 1 μg/μl in 2×SDS-reducing sample buffer. 30 μg of protein was loaded per lane and resolved by 4-20% SDS-PAGE. The following primary antibodies were used: mouse monoclonal anti-tau (Biosource), mouse monoclonal anti-β-tubulin isotype III (Sigma), mouse monoclonal anti-calmodulin (Upstate), rabbit anti-AMPAR1(GluR1) (Sigma), guinea pig anti-GLT-1 (Chemicon), rabbit anti-phospho-PKC_{α/β} (Cell Signaling), mouse anti-ATP5A1α (Molecular Probes), mouse anti-actin (Oncogene Res.) and mouse anti-His (Santa Cruz).

### (x) DNA Isolation

Oxidative adducts can form spontaneously with some DNA isolation protocols. To minimize ex vivo oxidation artifacts, genomic DNA was isolated from brain tissue and cultured cells by the silica-gel-membrane based DNeasy Tissue Kit (Qiagen) with the following modifications. To prevent oxidation, all buffers were purged with nitrogen and supplemented with 50 μM phenyl-tert-butyl nitrone (PBN) (Sigma), a free radical spin trap and scavenger. The high temperature incubation step was replaced by an extended incubation at 37° C. Following elution of purified DNA, 1 mM DTT was added prior to storage at −80° C.

### (xi) DNA Damage Assay

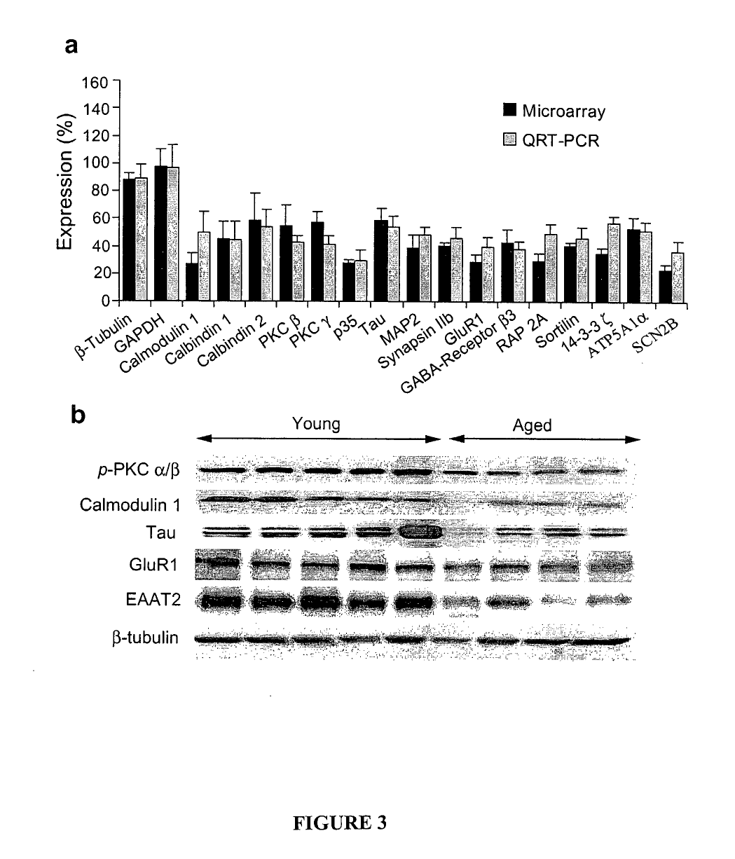

Formamidopyrimidine glycosylase (fpg) (New England Biolabs) is a bacterial endoglycoslase and AP-lyase that specifically excises 8-oxoguanine and other oxidized bases and creates a single strand break at the site of DNA damage. Quantitative real time PCR was used to determine the level of intact DNA in specific gene sequences before and after DNA cleavage by fpg. The fpg cleavage reaction was performed by incubating 250 ng of genomic DNA with 8 units of fpg in 1× NEBuffer 1 (10 mM Bis Tris Propane-HCl, 10 mM MgCl₂, 1 mM DTT, pH 7.0) and 100 μg/ml BSA in a volume of 50 μl. The fpg concentration and incubation time were predetermined according to an fpg dose response curve and time course. Under these conditions, an incubation time of 6-10 hrs is usually required for the reaction to reach steady state. Assays in this study were performed at 37° C. for 12 hr. Fpg enzyme was then inactivated by incubation at 60° C. for 5 min. The reaction mixture was then used for a quantitative PCR assay. In FIG. 3D, the following genes were analyzed. Age-stable genes: GAPDH, β-tubulin, ubiquitin B, MAP4, glutamate decarboxylase 2, internexin α, xeroderma pigmentosum G, and homer. Age-upregulated genes: non-selenium glutathione peroxidase (AOP2), low density lipoprotein receptor-related protein 4 (LRP4), secreted frizzled-related protein 1 (sFRP1), glycine amidinotransferase, TNFα, HIF1α, hOGG1 and S100. Age-downregulated genes: calmodulin 1, PKCγ, calcineurin Bα, sortilin, voltage-gated sodium channel IIβ (SCN2B), VAMP1, MAP2, CaM kinase IIα, Ca²⁺-ATPase (ATP2B2), calbindin 2, tau, GABA A receptor β3, synapsin 2, and mitochondrial F1 ATP synthase α (ATP5A1α).

(xii) Chromatin Immunoprecipitation

Incorporation of 8-oxo-guanine into genomic DNA was also assayed by chromatin immunoprecipitation (ChIP) with an anti-8-oxoguanine monoclonal antibody using the ChIP assay protocol (Upstate) with some modifications. Brain tissue samples (60 mg) were homogenized in Buffer A (10 mM HEPES-KOH pH 7.9 at 4° C., 1.5 mM MgCl₂, 10 mM KCl, 0.5 mM DTT, 1 mM EDTA, 1 mM EGTA, protease inhibitors and 1 mM PMSF) using a type B Dounce tissue grinder (Kontes). The homogenate was centrifuged at 500 rpm for 2 min to remove tissue fragments. Crude nuclei were collected by centrifugation at 3000 rpm (1000×g) for 10 min and resuspended in 360 ul Buffer B (10 mM HEPES, pH 7.5, 4 mM MgCl₂, 250 mM sucrose, and protease inhibitors). Chromatin was cross-linked by adding 10 μl 37% formaldehyde with rotation at 4° C. for 10 min and room temperature for 20 min. The reaction was stopped by adding 25 μl of 2 M glycine. After washing with ChIP Buffer B, the pellet was resuspended in 600 μl Lysis Buffer (1% SDS, 10 mM EDTA, 50 mM Tris-HCl, pH 8.1 and protease inhibitors) and sonicated with repeated 10 s pulses until the DNA was broken down to 500-600 bp fragments. Residual unfragmented chromatin was removed by centrifugation at 15,000×g for 10 min. The amount of DNA in the supernatant was quantified by measuring absorption at 260 nm, then adjusted to 100 ng/μl. 200 μl supernatant was diluted 10-fold in 2 ml ChIP dilution buffer (0.01% SDS, 1.1% Triton X-100, 1.2 mM EDTA, 16.7 mM Tris-HCl, pH 8.1, 167 mM NaCl, and protease inhibitors), and precleared twice with BSA-blocked Protein L Agarose (Pierce) (2×100 μl, 2×30 min at 4° C.). The beads were centrifuged and the supernatant was divided into 4×500 μl aliquots for immunoprecipitation, input DNA, and the IgG control. Primary antibody was added and incubated at 4° C. overnight. Mouse anti-8-oxoguanine monoclonal antibody (Chemicon) was used for immunoprecipitation of 8-oxoguanine, and ChromPure rabbit IgG (Jackson ImmunoResaerch) was used for the IgG control. 30 μl of BSA-blocked Protein L Agarose was then added and incubated at 4° C. with rotation. The beads were then centrifuged and washed once with a low salt immune complex buffer (Upstate), twice with a high salt wash buffer, once with a LiCl wash buffer (Upstate), and twice in TE buffer (10 mM Tris-HCl, 1 mM EDTA, pH 8.0). The washed agarose beads were eluted with 2×250 μl freshly prepared elution buffer (1% SDS, 0.1 mM NaHCO₃). DNA crosslinking was reversed by adding 5M NaCl and heating at 65° C. for 4 hrs. Protein was removed by incubation with 20 mg/ml proteinase K in 10 μM EDTA/40 mM Tris-HCl, pH 6.5 for 1 hr at 45° C. De-crosslinked DNA was then isolated by phenol/chloroform extraction and ethanol precipitation. The precipitated DNA was washed with 70% ethanol, air dried and dissolved in ddH₂O for PCR.

(xiii) Cell Culture

Human neuroblastoma SH-SY5Y cells were plated in 60 mm culture dishes at a density of 1.5×10⁶ cells per dish, and maintained in DMEM supplemented with 10% fetal bovine serum, 2 mM L-glutamine, 100 U/ml penicillin, and 100 μg/ml streptomycin. For differentiation, 2×10⁵ cells/well were grown in 6 well plates for 24 h, and then treated with 20 μM trans-retinoic-acid for 10 days (Smith (2002) Trends Cell Biol. 12, 28-36). The medium was changed every 3 days, and morphology was monitored until long neuritic processes were established. To induce oxidative DNA damage, 95% confluent cultures were treated with H₂O₂/FeCl₂ (300 μM/60 μM for undifferentiated cells; 150 μM/30 μM for differentiated cells) for the indicated time intervals. Cell viability determined by the MTS release assay (Promega) did not significantly change under the conditions used and was unaffected by hOGG1 overexpression. Fetal human cortical cultures were established by differentiation of human neuronal progenitor cells (Clonexpress) as described previously (Tu (1996) EMBO J. 15, 675-683). Cells were differentiated by adding 100 μM dibutyrtyl cAMP to the culture medium for at least 7 days until neuritic processes were established. Neuronal identity was confirmed by immunoreactivity for MAP2 and β-tubulin.

(xiiii) Luciferase Reporter Constructs

Promoter regions corresponding to the following sequences were cloned into the luciferase reporter vector pGL3-basic (Promega). β-Tubulin −617 to +79 (predicted); GAPDH −751 to +19 (Tchou et al. J. Biol. Chem. 269, 15318-15324 (1994); S100-533 to +41 (Harder, et al. Gene 113, 269-274 (1992); Tau −381 to +375 (Lindahl, T. & Barnes, D. E. Repair of endogenous DNA damage (2000) Cold Spring Harb. Symp. Quant. Biol. 65, 127-133; calmodulin 1-650 to +50 (Athas, et al. (1991) Cancer Res. 51, 5786-5793; Ca-ATPase −720 to +55 (predicted); Sortilin −543 to +38 (predicted). Promoter predictions were based on the human genome browser (Landfield et al. (1984) Science 226, 1089-1092 and the Neural Network Eukaryotic Promoter Prediction Tool.

### (vx) Knockdown of Mitochondrial ATP Synthase α

A 1 kb region of the ATP5A1α and topoisomerase IIβ genes without clear homology to other genes was amplified by RT-PCR and then transcribed into double stranded RNA (dsRNA) using the BLOCK-iT RNAi Transcription Kit (Invitrogen). dsRNA was processed further by Dicer into a pool of 21-23 nucleotide siRNA using the BLOCK-iT Dicer RNAi kit (Invitrogen). Both dsRNA and the final siRNA were verified by electrophoresis. ATP5A1α siRNA, the control topoisomerase IIβ siRNA, or a 21 nucleotide random oligonucleotide were transfected into SH-SY5Y cells using Lipofectamine 2000 (Invitrogen) and analyzed after 36 hours. ATP levels were determined using the luminescent signal based Cell Titer-Glo™ kit (Promega).

### Example 2

### Age-Dependent Regulation of Gene Expression

To investigate age-dependent regulation of gene expression in the human brain, RNA was harvested from postmortem samples of the frontal pole of 30 individuals ranging in age from 26 to 106 and was analysed using Affymetrix gene chips. To resolve genes with similar age-dependent expression patterns, the data was analysed for genes that correlate significantly with age and visualized by hierarchical clustering. This analysis demonstrated a cluster of co-regulated genes with reduced expression, and another cluster of genes with increased expression in aged individuals. To assess the rate of these gene changes, the entire transcriptome profile was compared at each age, and Pearson correlation coefficients were derived as a measure of similarity between any two ages. The group of individuals ≦42 years old showed the most homogeneous pattern of gene expression, and the group ≧73 years old was also relatively homogeneous (red colour indicating positive correlation, data not shown). Moreover, these two age groups were negatively correlated with each other (blue colour indicating negative correlation, data not shown). In contrast, the middle age group ranging in age from 45-71 exhibited much greater heterogeneity, with some cases resembling the young group and others resembling the aged group. These results suggest that a genetic signature of human cortical ageing may be defined starting in young adult life, and that the rate of age-related change may be heterogeneous among middle age individuals.

FIG. 3 shows DNA damage in the ageing human cortex. FIG. 3A is genomic DNA from fetal cortex does not exhibit significant DNA damage. DNA damage to the promoter regions of the indicated genes was assayed by cleavage with the endoglycosidase FPG and quantitative PCR. Intact DNA is the percentage detected by PCR following FPG cleavage relative to that in uncleaved DNA. FIG. 3B shows ageing increases oxidative DNA damage to the mitochondrial ATP synthase a (ATP5A1α) promoter. Shown are real-time fluorescence PCR curves from 26- and 77-year-old frontal cortical samples. Note the marked shift in PCR cycle number following FPG cleavage of 77 yr old DNA. FIG. 3C shows a time course of DNA damage in the ageing frontal cortex. DNA damage was assayed in the promoters of age-downregulated genes (calmodulin 1, Ca-ATPase, ATP5A1α, sodium channel 2β (SCN2B), VAMP1, and sortilin) in cortical samples from 26- to 106-year-old cases and normalized to the 26-year-old value (100%). Values represent the mean ±s.d.; n=3. Asterisks indicate intracortical biopsy samples. FIG. 3D shows DNA damage to promoters of genes that are stably expressed, downregulated or upregulated in the aged cortex. Shown is the fold increase in promoter DNA damage in aged cases (≧70 years old) relative to the youngest, 26-year-old case. Each point represents a gene. Asterisk indicates P<0.001 relative to age-stable genes by analysis of variance (ANOVA) with post-hoc Student-Newman-Keuls test. FIG. 3E shows oxidative damage to gene promoters in the aged cortex. Shown is the fold increase in 8-oxoguanine (8-oxo-dG) incorporation into promoters of age-stable (GAPDH, β-tubulin and synaptojanin 2), age-upregulated (S100), and age-downregulated genes (calmodulin 1 (CaM1), calbindin 1 (Calb1), calbindin 2 (Calb2), sortilin and PKCγ). Asterisks indicate P<0.05 relative to GAPDH. FIG. 3F shows chromatin immunoprecipitation of the calmodulin 1 promoter with a monoclonal antibody to 8-oxoguanine in aged (≧73-year-old) and young (<40-year-old) cortical samples. Input DNA and non-specific IgG (IgG) controls are shown.

### Example 3

### Quantitative Real-Time PCR Validation of the Microarray Data

Age-related genes were identified by performing statistical group comparison of frontal cortical samples from individuals ≦42 and ≧73 years old. About 4% of the approximately 1,000 genes analysed were significantly changed (1.5-fold or more, Table 3). To validate the microarray data, the quantitative real-time polymerase chain reaction (PCR) for a subset of functionally important genes, was compared. Microarray analysis and quantitative PCR generally showed consistent changes (FIG. 3A). The confirmation of microarray results for synaptic, calcium homeostasis and transport-related gene s is shown in FIG. 3A where mRNA levels of selected genes in the aged frontal cortex determined by microarray analysis and quantitative RT-PCR. Values are percentage mRNA levels in aged cases (≧73 years old) versus young cases (≦42 years old) and represent the mean ±s.d.; n=4. Furthermore, consistent changes at the protein level were observed for a subset of genes analysed by western blotting. FIG. 3B shows immunoblots from five young and four aged frontal cortical samples. The p-PKCα/β blot specifically resolves activated phosphorylated forms of PKCα/β. EAAT2 is the predominant human brain glutamate transporter. The postmortem interval did not correlate significantly with the messenger RNA expression levels of 40 age-regulated genes examined, or with a cumulative measure of all the genes in each of the two age-related clusters. In addition, expression of a number of neuron-specific markers, including β-tubulin, contactin 2 (TAG-1), GAP-43, γ-enolase, and syntaxin 1, did not change significantly with age, suggesting that ageing was not associated with major changes in neuronal cell number.

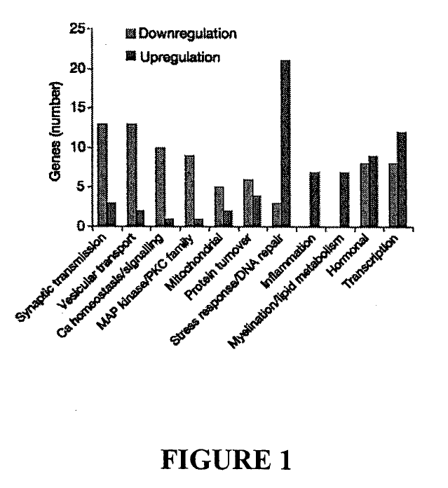

Genes that play a role in synaptic function and the plasticity that underlies learning and memory were among those most significantly affected in the ageing human cortex (Table 1, FIGS. 1 and 3). Several neurotransmitter receptors that are centrally involved in synaptic plasticity (Kandel, et al. (2001) Science 294, 1030-1038 and Malinow, et al. (2002) Annu. Rev. Neurosci. 25, 103-126 showed significantly reduced expression after age 40, including the GluR1 AMPA (α-amino-3-hydroxy-5-methyl-4-isoxazole propionic acid) receptor subunit, the NMDA (N-methyl-D-aspartate) R2A receptor subunit, and subunits of the GABA_{A} receptor. Moreover, the expression of genes that mediate synaptic vesicle release and recycling was significantly reduced, notably VAMP1/synaptobrevin, synapsin II, RAB3A and SNAPs.

Members of the major signal transduction systems that mediate long-term potentiation (LTP) and memory storage were age-downregulated, notably the synaptic calcium signalling system, with reduced expression of calmodulin 1 and CAM kinase IIα (Table 1 and FIGS. 3A and 3B). The major calcium-binding proteins calbindins 1 and 2, the calcium pump ATP2B2, and the calcium-activated transcription factor MEF2C that promotes neuronal survival were also significantly reduced (Mao et al. (1999) Science 286, 785-790 and Okamoto, et al. (2000) Proc. Natl. Acad. Sci. USA 97: 7561-7566). Furthermore, multiple members of the protein kinase C (PKC) and Ras-MAP (mitogen-activated protein) kinase signalling pathways showed decreased expression. The activation state of PKC was also reduced, as indicated by decreased levels of activated phosphorylated forms (FIG. 3B). Thus, calcium homeostasis and neuronal signalling may be affected in the aged cortex.

Genes involved in vesicular/protein transport showed reduced expression in the aged cortex, including multiple RAB GTPases, sortilin, dynein, and clathrin light chain (Table 1). Moreover, microtubule-associated proteins (MAP1B, MAP2, tau and kinesin 1B) that stabilize microtubules and promote axonal transport were consistently and robustly reduced. The p35 activator of cyclin-dependent kinase-5 (cdk5), which regulates intran euronal protein trafficking and synaptic function was also significantly reduced (Smith et al. (2002) Trends Cell Biol. 12, 28-36). Thus, vesicular trafficking may be affected in the aged human cortex. In addition, a number of genes involved in protein turnover also showed reduced expression in aged cortex, including ubiquitin-conjugating enzymes, the lysosomal proton pump, and the enzymes D-aspartate O-methyltransferase and methionine adenosyltransferase II, which repair damaged proteins.

The ageing of the human frontal cortex was also associated with increased expression of genes that mediate stress responses and repair (FIG. 1 and Table 1). These included genes involved in protein folding (heat shock protein 70 and α crystallin), antioxidant defence (nonselenium glutathione peroxidase, paraoxonase and selenoprotein P) and metal ion homeostasis (metallothioneins 1B, 1G and 2A). Genes involved in inflammatory or immune responses, such as tumour-necrosis factor (TNF)-α, were also increased. Increased expression of the base-excision repair enzymes 8-oxoguanine DNA glycosylase and uracil DNA glycosylase is consistent with increased oxidative DNA damage in the aged cortex.

TABLE 1
Age-regulated genes in the human frontal cortex

| Function | Gene name | Accession number | Fold y | q value |
|---|---|---|---|---|
| Synaptic function | | | | |
| Synaptic transmission | GluR1 | M81886 | 2.2 to 2.4 | 0.002 |
| | NMDA receptor 2A | U09002 | 2.3 | 0.002 |
| | GABA A receptor ÿ | M82919 | 3.2 | 0.002 |
| | GABA A receptor ÿ | AF016917 | 1.5 | 0.002 |
| | Serotonin receptor 2A | AA418537 | 2.0 | 0.002 |
| | Voltage-gated Na channel II ÿ (SCN2B) | AF049498 | 5.1 | 0.002 |
| | Voltage-dependent calcium channel ÿ2 | U95019 | 1.9 | 0.002 |
| | Neurexin 1 | AB011150 | 1.6 | 0.002 |
| | Synaptobrevin 1 (VAMP1) | M36200 | 3.4 | 0.002 |
| | Synapsin II b | U40215 | 3.4 | 0.002 |
| | ySNAP | U78107 | 2.2 | 0.002 |
| | ySNAP | U39412 | 1.6 | 0.005 |
| | RAB3A | M28210 | 1.7 | 0.002 |
| | SNAP23 | AJ011915 | 1.7 | 0.005 |
| | Synaptophysin-like protein | X68194 | 1.8 | 0.006 |
| Ca²⁺ homeostasis/signalling | Calmodulin 1 | U12022 | 2.2 to 4.1 | 0.002 |
| | Calmodulin 3 | J04046 | 1.6 | 0.002 |
| | Calbindin 1 (28 kD) | AF068862 | 2.5 | 0.002 |
| | Calbindin 2 (29 kD, calretinin) | X56667 | 1.6 | 0.003 |
| | CaM kinase II ÿ | AB023185 | 1.7 | 0.008 |
| | CaM kinase IV | D30742 | 2.0 | 0.007 |
| | Calcineurin B ÿ | M30773 | 2.8 | 0.002 |
| | ATPase, Ca²⁺-transporting, plasma membrane 2 (ATP2B2) | L20977 | 2.5 | 0.002 |
| | ATPase, Ca²⁺-transporting, plasma membrane 2 (ATP2A2) | M23114 | 1.6 | 0.002 |
| | Regucalcin (senescence marker protein) | D31815 | 1.7 | 0.002 |
| cAMP signaling | Phosphodiesterase 4D | U02882 | 1.9 | 0.002 |
| | Adenylyl cyclase associated protein 2 | HG2530 | 1.6 to 2.3 | 0.002- |
| | | | | 0.003 |
| Protein kinase C | PKCy | X06318 | 1.9 to 2.9 | 0.002 |
| | PKCy | Z15114 | 1.8 | 0.002 |
| | PKCy | Z15108 | 1.7 | 0.002 |
| G protein signalling | Rap2A | X12534 | 3.8 to 4.1 | 0.002 |
| | Regulator of G protein signalling 4 | U27768 | 1.8 to 2.2 | 0.002 |
| | G protein, q polypeptide (GNAQ) | U43083 | 2.0 | 0.002 |
| MAP kinase cascades | MAPK1 | Z11695 | 1.9 | 0.002 |
| | MAPK9 | U09759 | 1.7 | 0.003 |
| | MAPKK4 | U17743 | 3.1 | 0.002 |
| | RasGNRF | HG2510 | 2.4 to 4.7 | 0.003 |
| | | | | 0.008 |
| | MAPKK5 | U67156 | 1.6 | 0.002 |
| | 14-3-3 y | U28964 | 3.6 | 0.002 |
| | p21 activated protein kinase (PAK1) | U24152 | 2.7 | 0.002 |
| CdK5 | CdK5, regulatory subunit 1 (p35) | X80343 | 3.4 | 0.002 |
| Vesicular transport | RAB1A | M28209 | 1.6 | 0.006 |
| | RAB3A | M28210 | 1.7 | 0.002 |
| | RAB5A | M28215 | 1.9 | 0.002 |
| | RAB6A | M28212 | 3.5 | 0.002 |
| | Kinesin 1B | AB011163 | 2.2 | 0.002 |
| | Sortilin 1 | X98248 | 3.5 | 0.002 |
| | Dynein (DNCH1) | H05552 | 2.4 | 0.002 |
| | Dynamin 1-like | AF000430 | 1.6 | 0.002 |
| | Trans Golgi network protein 2 | AF027516 | 2.2 | 0.002 |
| | Golgi reassembly stacking protein 2 | W26854 | 1.7 | 0.002 |
| | Phosphotidylinositol transfer protein ÿ | D30037 | 2.0 | 0.002 |
| | Clathrin, light polypeptide | M20470 | 1.6 | 0.002 |
| | Kinesin 2 | L04733 | 1.7 | 0.005 |
| | VAMP3 | H93123 | 1.5 | 0.002 |
| Microtubule cytoskeleton | MAP1B | L06237 | 4.9 | 0.002 |
| | MAP2 | U01828 | 2.1 to 4.2 | 0.002 |
| | Tau | J03778 | 2.3 | 0.002 |
| | RAN binding protein 9 | AF064606 | 1.7 | 0.005 |
| Neuronal survival | MADS box transcription enhancer factor 2C (MEF2C) | S57212 | 2.7 | 0.002 |
| | Inositol polyphosphate-4-phosphatase I | AI955897 | 2.0 | 0.002 |
| | Inositol 1,4,5 trisphosphate 3 kinase A | X54938 | 2.5 | 0.002 |
| | Inositol 1,4,5 trisphosphate 3-kinase B | X57206 | 1.9 | 0.002 |
| Protein turnover | ATPase, H⁺-transporting, lysosomal V1 subunit H | W27838 | 2.5 | 0.005 |
| | ATPase, H⁺-transporting, lysosomal V1 subunit A | L09235 | 1.7 | 0.007 |
| | ATPase, H⁺-transporting, lysosomal V1 subunit G 2 | W26326 | 1.5 | 0.002 |
| | Ubiquitin conjugating enzyme Ubch5 | HG3344 | 1.6 | 0.002 |
| | Ubiquitin conjugating enzyme E2M | AF075599 | 1.6 | 0.002 |
| | Ubiquitin carrier protein | M91670 | 1.6 | 0.002 |
| | Lysosomal associated membrane protein 2 | U36336 | 2.3 | 0.002 |
| | Calpastatin (calpain inhibitor) | D16217 | 1.6 | 0.007 |
| | Serine/cysteine proteinase inhibitor | D83174 | 1.7 | 0.005 |
| | Angiotensinogen (serine/cysteine) proteinase Inhibitor A8 | K02215 | 1.6 | 0.002 |
| Amino acid modification | Protein-L-isoaspartate (Daspartate) O-methyltransferase | D25547 | 2.7 | 0.002 |
| | Methionine adenosyltransferase IIÿ | X68836 | 2.1 | 0.002 |
| | Beta-1,3-galactosyltransferase | Y15062 | 1.9 | 0.002 |
| | Glutamate decarboxylase 1 | M81883 | 1.6 | 0.005 |
| | Methionine synthase reductase | AF025794 | 1.6 | 0.008 |
| | Transglutaminase 2 | M55153 | 2.8 | 0.002 |
| | Glycine amidinotransferase | S68805 | 1.5 to 1.8 | 0.002 |
| | Lysine hydroxylase 2 | U84573 | 2.4 | 0.002 |
| Mitochondrial | ATP synthase, H⁺-transporting, mitochondrial Flÿ | D14710 | 2.3 | 0.002 |
| | Mitochondrial ribosomal protein L28 | U19796 | 1.7 | 0.002 |
| | Mitochondrial ribosomal protein S12 | Y11681 | 2.2 | 0.002 |
| | Cytochrome c synthase | U36787 | 1.6 | 0.002 |
| | Translocase of inner mitochondrial membrane 17 A | X97544 | 2.0 | 0.002 |
| | Monoamine oxidase A | AA420624 | 1.6 | 0.002 |
| | Mitochondrial 3-oxoacyl-Coenzyme A thiolase | D16294 | 1.5 | 0.003 |
| Stress response | | | | |
| Antioxidant | Nonselenium glutathione peroxidase | D14662 | 1.7 | 0.002 |
| | Selenoprotein P | Z11793 | 1.7 | 0.002 |
| | Paraoxonase 2 | AF001601 | 1.6 | 0.002 |
| | Cystathionine-beta-synthase | L00972 | 1.6 | 0.002 |
| DNA repair | 8-oxoguanine DNA glycosylase | U88620 | 1.6 | 0.006 |
| | Uracil-DNA glycosylase | Y09008 | 1.7 | 0.005 |
| | Topoisomerase 1 binding protein | U82939 | 1.6 | 0.009 |
| | Topoisomerase II ÿ | M27504 | 1.7 | 0.003 |
| | FK506 binding protein 12-rapamycin associated protein 1 | L34075 | 1.9 | 0.002 |
| Stress | Heat shock 70 kD protein 2 | L26336 | 1.9 to 2.2 | 0.005-.006 |
| | Crystallin, alpha B | AL038340 | 1.6 to 2.0 | 0.002- |
| | | | | 0.003 |
| | Hypoxia inducible factor 1 ÿ (HIF1 ÿ) | U22431 | 2.0 | 0.005 |
| | HIF-1 responsive RTP801 | AA522530 | 2.5 | 0.002 |
| | Transglutaminase 2 | M55153 | 2.8 | 0.002 |
| | p53 binding protein 2 | U58334 | 1.7 | 0.002 |
| | Retinoblastoma-associated protein 140 | AB029028 | 1.8 | 0.007 |
| | Retinoblastoma-like 2 (p130) | X76061 | 1.6 | 0.007 |
| | Stress 70 protein chaperone | U04735 | 1.8 | 0.006 |
| Metal ion homeostasis | Metallothionein 1G | J03910 | 2.2 | 0.005 |
| | Metallothionein 1B | M13485 | 1.6 | 0.002 |
| | Metallothionein 2A | R92331 | 1.5 to 1.7 | 0.002 |
| | Haem binding protein 2 | W27949 | 2.0 | 0.002 |
| | Haemoglobin ÿ | L48215 | 2.9 to 3.3 | 0.002 |
| | Hephaestin | AB014598 | 1.6 | 0.002 |
| Inflammation | TNF-ÿ | AF010312 | 2.7 | 0.006 |
| | C type lectin | X96719 | 2.7 | 0.003 |
| | H factor (complement)-1 | M65292 | 3.2 | 0.002 |
| | Interferon, gamma-inducible protein 16 | M63838 | 2.0 | 0.005 |
| | Interferon regulatory factor 7 | U53831 | 1.9 | 0.003 |
| | Integrin ÿ | M14648 | 1.8 | 0.002 |
| | Integrin ÿ1 | X07979 | 1.7 | 0.002 |
| Myelination/lipid metabolism | Oligodendrocyte lineage transcription factor 2 | U48250 | 1.7 | 0.002 |
| | Peripheral myelin protein 22 | D11428 | 1.7 | 0.003 |
| | Proteolipid protein 1 | M54927 | 1.6 | 0.002 |
| | Fatty acid desaturase 1 | AF009767 | 1.8 to 2.1 | 0.002 |
| | Apolipoprotein D | J02611 | 2.1 | 0.002 |
| | Low density lipoprotein receptor related protein 4 | AB011540 | 2.0 | 0.002 |
| | Sterol carrier protein 2 | U11313 | 1.6 | 0.002 |
| | Phospholipase D3 | U60644 | 1.6 | 0.002 |
| Transcription | Transcription factor ZHX2 | AB020661 | 2.1 | 0.002 |
| | NK2 transcription factor | AF019415 | 1.5 to 1.8 | 0.005- |
| | | | | 0.008 |
| | Inhibitor of DNA binding 4 (ID4) | AL022726 | 2.3 | 0.002 |
| | Zinc finger protein 238 | U38896 | 4.6 | 0.002 |
| | Forkhead box G1A | X74143 | 2.0 | 0.002 |
| | Chromatin remodelling complex (SMARCC2) | D26155 | 1.8 | 0.002 |
| | ETS2 | J04102 | 1.7 | 0.002 |
| | E2F transcription factor 4 | S75174 | −1.6 | 0.003 |
| Hormonal | Insulin receptor | X02160 | 1.6 | 0.004 |
| | Leptin receptor | AW026535 | 1.7 | 0.002 |
| | Orexin receptor | AF041245 | 1.6 | 0.002 |
| | Vascular endothelial growth factor | AF022375 | 1.8 | 0.005 |
| | Secreted frizzled related protein 1 | AF056087 | 1.9 | 0.009 |
| | FGF receptor 2 | M87770 | 1.6 | 0.002 |
| | FGF receptor 3 | M64347 | 1.8 | 0.002 |
| | FGF2 (basic) | J04513 | 2.1 | 0.002 |
| | Proenkephalin | J00123 | 2.5 | 0.003 |
| | Somatostatin | AI636761 | 1.8 to 2.9 | 0.002 |
| | Cholecystokinin B receptor | L10822 | 2.7 | 0.002 |
| | Chromogranin B (secretogranin 1) | Y00064 | 1.6 | 0.007 |
| | RevErbA ÿ receptor (NR1D2) | D16815 | 2.9 | 0.003 |
| | GDNF receptor ÿ | AF002700 | 1.6 | 0.003 |
| | FGF 12 | AL119322 | 2.4 to 2.6 | 0.002-.008 |
| | FGF 13 | U66198 | 2.3 to 3.2 | 0.002-.005 |

Shown are selected age-regulated genes representative of functional groups. Age-downregulated genes are blue and age-upregulated genes are red. Fold changes and statistical q values with a range reflect multiple probe sets for the same gene. Gene accession numbers are provided. See Supplementary Table 2 for a complete list of age-regulated genes.

TABLE 2
Human Brain Samples

| Age (yr) | Gender | PMI (hr) | Age (yr) | Gender | PMI (hr) |
|---|---|---|---|---|---|
| 26 | M | 8 | 61 | F | 4 |
| 26B | M | 21 | 61* | M | biopsy |
| 27 | F | 18 | 66 | M | 6 |
| 29 | M | 18 | 70 | M | 2 |
| 30 | F | 7 | 71 | F | 9 |
| 31* | F | biopsy | 73 | M | 12 |
| 36 | F | 6 | 77 | M | 1 |
| 37 | M | 18 | 80 | M | 4 |
| 38 | F | 4 | 81 | F | 1 |
| 40 | M | 16 | 85 | M | 2 |
| 42 | M | 8 | 87 | F | 4 |
| 45 | M | 17 | 90 | F | 13 |
| 48 | F | 18 | 90B | M | 5 |
| 52 | M | 6 | 91 | M | 7 |
| 53 | M | 18 | 95 | M | 19 |
| 56 | F | biopsy | 106 | F | 12 |

PMI: postmortem interval.
*Used only for DNA damage assay due to limited tissue

TABLE 3
Age-Regulated Genes

| Gene Name | Accession Number | Fold Change | q-value |
|---|---|---|---|
| 14-3-3 epsilon | U54778 | −1.59 | 0.00214 |
| 14-3-3 zeta | U28964 | −3.61 | 0.00214 |
| 3-hydroxysteroid epimerase | U89281 | 1.54 | 0.00648 |
| 50h7 Homo sapiens cDNA | W28732 | −2.08 | 0.00214 |
| 5-hydroxytryptamine (serotonin) receptor 2A | AA418537 | −2.04 | 0.00214 |
| 5-methyltetrahydrofolate-homocysteine methyltransferase reductase | AF025794 | 1.57 | 0.00879 |
| 5′-nucleotidase, cytosolic II | D38524 | 1.62 | 0.00992 |
| 8-oxoguanine DNA glycosylase | U88620 | 1.59 | 0.00648 |
| acetyl-Coenzyme A acyltransferase 2 | D16294 | 1.54 | 0.00381 |
| achaete-scute complex-like 1 (Drosophila) | L08424 | 1.7 | 0.00214 |
| acidic (leucine-rich) nuclear phosphoprotein 32B | Y07969 | 1.65 | 0.00381 |
| actin, alpha 1, skeletal muscle | J00068 | −1.89 | 0.00214 |
| adducin 3 (gamma) | D67031 | 1.81 | 0.00214 |
| Adenylyl Cyclase-Associated Protein 2 | HG2530-HT2626 | −2.34 | 0.00214 |
| Adenylyl Cyclase-Associated Protein 2 | HG2530-HT2626 | −1.55 | 0.00381 |
| adipose specific 2 | AI381790 | 1.67 | 0.00648 |
| aldehyde dehydrogenase 4 family, member A1 | U24266 | 1.56 | 0.00214 |
| aldehyde dehydrogenase 9 family, member A1 | U34252 | 1.58 | 0.00214 |
| alpha globin gene cluster on chromosome 16-zeta | J00153 | 3.19 | 0.00214 |
| alpha-actinin-2-associated LIM protein | AF002282 | 1.95 | 0.00214 |
| angiomotin | AB028994 | 1.61 | 0.00214 |
| angiomotin like 2 | AB023206 | 1.56 | 0.00992 |
| angiotensinogen (serine (or cysteine) proteinase inhibitor, clade A8 | K02215 | 1.59 | 0.00214 |
| ankyrin 2, neuronal | X56958 | −2.14 | 0.00214 |
| annexin A3 | M20560 | 2.27 | 0.00512 |
| annexin A5 | U05770 | 1.56 | 0.00761 |
| apolipoprotein D | J02611 | 2.08 | 0.00214 |
| ATP synthase, H+ transporting, mitochondrial F1a | D14710 | −2.27 | 0.00214 |
| ATPase, aminophospholipid transporter (APLT), Class I, type 8A, | AB013452 | −1.84 | 0.00214 |
| member 1 | | | |
| ATPase, Ca++ transporting, cardiac muscle, slow twitch 2 | M23114 | −1.55 | 0.00214 |
| ATPase, Ca++ transporting, plasma membrane 2 | L20977 | −2.55 | 0.00214 |
| ATPase, Ca++ transporting, plasma membrane 2 | X63575 | −2.61 | 0.00214 |
| ATPase, H+ transporting, lysosomal 13 kDa, V1 subunit G isoform 2 | W26326 | −1.51 | 0.00214 |
| ATPase, H+ transporting, lysosomal 50/57 kDa, V1 subunit H | W27838 | −2.51 | 0.00512 |
| ATPase, H+ transporting, lysosomal 70 kDa, V1 subunit A | L09235 | −1.7 | 0.00761 |
| ATP-binding cassette, sub-family D (ALD), member 3 | X83467 | 1.64 | 0.00512 |
| ATP-binding cassette, sub-family G (WHITE), member 1 | X91249 | 1.77 | 0.00512 |
| BAF53 | AF041474 | 1.93 | 0.00214 |
| basic transcription factor 2 | AL080209 | 1.79 | 0.00992 |
| B-cell CLL/lymphoma 11A (zinc finger protein) | W27619 | −2.69 | 0.00214 |
| B-cell CLL/lymphoma 2 | M14745 | 1.67 | 0.00214 |
| bone morphogenetic protein 7 (osteogenic protein 1) | X51801 | 1.64 | 0.00214 |
| brain-specific protein p25 alpha | AB017016 | −2 | 0.00214 |
| bromodomain adjacent to zinc finger domain, 2B | AL080173 | 1.64 | 0.00761 |
| bromodomain containing 2 | S78771 | −1.94 | 0.00648 |
| cadherin-like 22 | AF035300 | −1.61 | 0.00214 |
| calbindin 1, 28 kDa | AF068862 | −2.5 | 0.00214 |
| calbindin 2, 29 kDa (calretinin) | X56667 | −1.57 | 0.00381 |
| calcium channel, voltage-dependent, beta 2 subunit | U95019 | −1.94 | 0.00214 |
| calcium/calmodulin-dependent protein kinase (CaM kinase) II alpha | AB023185 | −1.74 | 0.00879 |
| calcium/calmodulin-dependent protein kinase IV | D30742 | −1.98 | 0.00761 |
| caldesmon 1 | M64110 | 1.86 | 0.00512 |
| calmodulin 1 (phosphorylase kinase, delta) | U12022 | −4.06 | 0.00214 |
| calmodulin 1 (phosphorylase kinase, delta) | W28510 | −2.18 | 0.00214 |
| calmodulin 3 (phosphorylase kinase, delta) | J04046 | −1.6 | 0.00214 |
| calpastatin | D16217 | 1.6 | 0.00761 |
| calpastatin | U31346 | 1.62 | 0.00992 |
| cAMP-regulated guanine nucleotide exchange factor II | U78516 | 1.54 | 0.00879 |
| CAP, adenylate cyclase-associated protein, 2 (yeast) | U02390 | −1.59 | 0.00214 |
| carbohydrate (chondroitin) synthase 1 | AB023207 | 1.84 | 0.00381 |
| carbonic anhydrase IV | M83670 | −1.51 | 0.00648 |
| caveolin 1, caveolae protein, 22 kDa | AF070648 | 1.57 | 0.00214 |
| caveolin 2 | AF035752 | 1.65 | 0.00761 |
| CDC-like kinase 1 | M59287 | 1.57 | 0.00879 |
| cell division cycle 2-like 2 | M37712 | 2.22 | 0.00214 |
| cell division cycle 40 homolog (yeast) | AF038392 | −4.74 | 0.00512 |
| centrin, EF-hand protein 3 | AI056696 | 1.72 | 0.00214 |
| chloride intracellular channel 2 | Y12696 | 1.7 | 0.00992 |
| chloride intracellular channel 4 | AL080061 | 1.95 | 0.00512 |
| cholecystokinin B receptor | L10822 | −2.69 | 0.00214 |
| chondroitin sulfate proteoglycan 2 (versican) | X15998 | 2.5 | 0.00214 |
| chondroitin sulfate proteoglycan 2 (versican) | X15998 | 1.63 | 0.00214 |
| chondroitin sulfate proteoglycan 6 (bamacan) | AF020043 | 1.79 | 0.00381 |
| chromogranin B (secretogranin 1) | Y00064 | −1.56 | 0.00761 |
| chromosome 14 open reading frame 147 | AL080066 | 1.88 | 0.00214 |
| clathrin, light polypeptide (Lcb) | M20470 | −1.63 | 0.00214 |
| claudin 5 | AF000959 | 1.52 | 0.00648 |
| cleavage and polyadenylation specific factor 5, 25 kDa | AJ001810 | −2.24 | 0.00214 |
| collagen, type IV, alpha 5 (Alport syndrome) | M58526 | 1.76 | 0.00879 |
| contactin 1 | Z21488 | −2.52 | 0.00214 |
| copine III | AB014536 | 1.88 | 0.00214 |
| coronin, actin binding protein, 1A | D44497 | −1.67 | 0.00214 |
| corticotropin releasing hormone | V00571 | −2.75 | 0.00214 |
| crystallin, alpha B | AL038340 | 1.99 | 0.00214 |
| crystallin, alpha B | AL038340 | 1.6 | 0.00381 |
| C-type lectin, superfamily member 2 | X96719 | 2.71 | 0.00381 |
| cutaneous T-cell lymphoma-associated tumor antigen se20-4 | AB015345 | −1.77 | 0.00214 |
| cyclin-dependent kinase 5, regulatory subunit 1 (p35) | X80343 | −3.41 | 0.00214 |
| cystathionine-beta-synthase | L00972 | 1.59 | 0.00214 |
| cysteine and glycine-rich protein 1 | M33146 | 1.55 | 0.00214 |
| cytochrome P450, family 1, subfamily B, polypeptide 1 | U03688 | 1.78 | 0.00992 |
| DEAD (Asp-Glu-Ala-Asp) box polypeptide 3, X-linked | U50553 | −1.85 | 0.00214 |
| deoxyribonuclease I-like 1 | X90392 | 1.64 | 0.00381 |
| dihydropyrimidinase-like 3 | D78014 | 1.83 | 0.00214 |
| discoidin domain receptor family, member 1 | L20817 | 1.82 | 0.00214 |
| DKFZP434J214 protein | AL080156 | 1.88 | 0.00214 |
| DKFZP564O0823 protein | AL080121 | −1.71 | 0.00214 |
| DKFZP586A0522 protein | AL050159 | 1.79 | 0.00214 |
| dual specificity phosphatase 3 (vaccinia virus phosphatase VH1-related) | L05147 | −1.76 | 0.00214 |
| dual specificity phosphatase 3 (vaccinia virus phosphatase VH1-related) | L05147 | −1.65 | 0.00214 |
| DVS27-related protein | AB024518 | 2.74 | 0.00512 |
| dynamin 1-like | AF000430 | −1.56 | 0.00214 |
| dynein, cytoplasmic, intermediate polypeptide 1 | H05552 | −2.45 | 0.00214 |
| E1A binding protein p400 | U80743 | −1.51 | 0.00761 |
| E2F transcription factor 4, p107/p130-binding | S75174 | −1.56 | 0.00381 |
| EGF-containing fibulin-like extracellular matrix protein 1 | U03877 | 2.63 | 0.00214 |
| EGF-like-domain, multiple 4 | AB011541 | −2.26 | 0.00214 |
| ELAV-like 2 (Hu antigen B) | U12431 | −1.73 | 0.00214 |
| elongation of very long chain fatty acids like 2 | AL080199 | 1.53 | 0.00381 |
| embryonal Fyn-associated substrate | AB001466 | 2.58 | 0.00512 |
| endosulfine alpha | X99906 | −1.79 | 0.00214 |
| endosulfine alpha | X99906 | −1.65 | 0.00512 |
| endothelial differentiation, sphingolipid G-protein-coupled receptor, 1 | M31210 | 2.35 | 0.00214 |
| epilepsy, progressive myoclonus type 2A, Lafora disease (laforin) | AF084535 | 1.68 | 0.00761 |
| extracellular matrix protein 2, female organ and adipocyte specific | AB011792 | 1.63 | 0.00512 |
| eyes absent homolog 1 (Drosophila) | AJ000098 | 1.61 | 0.00381 |
| fatty acid desaturase 1 | AF009767 | 1.85 | 0.00214 |
| fatty acid desaturase 1 | AF009767 | 2.1 | 0.00214 |
| fer-1-like 3, myoferlin (C. elegans) | AL096713 | 2.15 | 0.00214 |
| FGF | X59065 | 2.19 | 0.00214 |
| fibroblast growth factor 12 | AL119322 | −2.59 | 0.00214 |
| fibroblast growth factor 12 | AA169447 | −2.39 | 0.00879 |
| fibroblast growth factor 13 | U66198 | −2.33 | 0.00214 |
| fibroblast growth factor 13 | U66198 | −3.25 | 0.00512 |
| fibroblast growth factor 2 (basic) | J04513 | 2.1 | 0.00214 |
| fibroblast growth factor 2 (basic) | J04513 | 2.08 | 0.00214 |
| fibroblast growth factor receptor 2 | M87770 | 1.64 | 0.00214 |
| fibroblast growth factor receptor 3 | M64347 | 1.78 | 0.00214 |
| FK506 binding protein 12-rapamycin associated protein 1 | L34075 | −1.94 | 0.00214 |
| Fk506-Binding Protein, Alt. Splice 2 | HG1139-HT4910 | −2.74 | 0.00214 |
| follicular lymphoma variant translocation 1 | X63657 | 1.95 | 0.00214 |
| forkhead box G1A | X74143 | −1.97 | 0.00214 |
| FUS interacting protein (serine-arginine rich) 1 | AF047448 | −1.81 | 0.00761 |
| FXYD domain containing ion transport regulator 1 (phospholemman) | AA524547 | 1.61 | 0.00214 |
| FYN oncogene related to SRC, FGR, YES | Z97989 | 1.93 | 0.00214 |
| G protein-coupled receptor, family C, group 5, member B | AC004131 | 1.66 | 0.00214 |
| gamma-aminobutyric acid (GABA) A receptor, beta 3 | M82919 | −3.16 | 0.00214 |
| gamma-aminobutyric acid (GABA) A receptor, delta | AF016917 | −1.54 | 0.00214 |
| gamma-butyrobetaine hydroxylase 1 | AF082868 | 1.92 | 0.00214 |
| gap junction protein, alpha 1, 43 kDa (connexin 43) | X52947 | 1.68 | 0.00214 |
| GDNF family receptor alpha 2 | AF002700 | −1.64 | 0.00381 |
| gelsolin (amyloidosis, Finnish type) | X04412 | 1.55 | 0.00214 |
| glial fibrillary acidic protein | S40719 | 1.61 | 0.00214 |
| glutamate decarboxylase 1 (brain, 67 kDa) | M81883 | −1.61 | 0.00512 |
| glutamate receptor, ionotropic, AMPA 1 | M64752 | −2.41 | 0.00214 |
| glutamate receptor, ionotropic, AMPA 1 | M81886 | −2.22 | 0.00214 |
| glutamate receptor, ionotropic, N-methyl D-aspartate 2A | U09002 | −2.34 | 0.00214 |
| glycine amidinotransferase (L-arginine:glycine amidinotransferase) | S68805 | 1.53 | 0.00214 |
| glycine amidinotransferase (L-arginine:glycine amidinotransferase) | S68805 | 1.83 | 0.00214 |
| glypican 5 | U66033 | 1.59 | 0.00761 |
| golgi reassembly stacking protein 2, 55 kDa | W26854 | −1.69 | 0.00214 |
| GRB2-associated binding protein 2 | AB011143 | 2.05 | 0.00992 |
| GREB1 protein | AB011147 | 1.81 | 0.00512 |
| guanine nucleotide binding protein (G protein), beta 5 | AF017656 | −1.68 | 0.00761 |
| guanine nucleotide binding protein (G protein), gamma 11 | U31384 | 2.11 | 0.00214 |
| guanine nucleotide binding protein (G protein), gamma 12 | AL049367 | 1.99 | 0.00214 |
| guanine nucleotide binding protein (G protein), q polypeptide | U43083 | −2.03 | 0.00214 |
| Guanine Nucleotide-Binding Protein Rap2, Ras-Oncogene Related | HG1996-HT2044 | −4.78 | 0.00214 |
| H factor (complement)-like 1 | M65292 | 3.17 | 0.00214 |
| haptoglobin | X89214 | 1.73 | 0.00879 |
| heat shock 70 kDa protein 2 | L26336 | 2.22 | 0.00512 |
| heat shock 70 kDa protein 2 | L26336 | 1.88 | 0.00648 |
| heat shock 70 kDa protein 8 | Y00371 | −1.56 | 0.00214 |
| Heat Shock Protein, 70 Kda | HG2855-HT2995 | −1.56 | 0.00214 |
| heme binding protein 2 | W27949 | 1.98 | 0.00214 |
| hemoglobin, beta | L48215 | 2.87 | 0.00214 |
| hemoglobin, beta | M25079 | 3.29 | 0.00214 |
| hephaestin | AB014598 | 1.58 | 0.00214 |
| heterogeneous nuclear ribonucleoprotein H3 (2H9) | AF052131 | 1.51 | 0.00214 |
| HIF-1 responsive RTP801 | AA522530 | 2.55 | 0.00214 |
| histone 1, H2ac | U90551 | 1.58 | 0.00381 |
| holocytochrome c synthase (cytochrome c heme-lyase) | U36787 | −1.61 | 0.00214 |
| Homo sapiens BCE-1 mRNA, complete cds | AF068197 | 1.57 | 0.00992 |
| Homo sapiens cDNA FLJ13267 fis, clone OVARC1000964. | D45288 | 1.98 | 0.00648 |
| Homo sapiens cDNA FLJ45029 fis, clone BRAWH3018326 | H12054 | −1.69 | 0.00214 |
| Homo sapiens LOC340111 (LOC340111), mRNA | X75940 | 2.28 | 0.00992 |
| Homo sapiens LOC345780 (LOC345780), mRNA | AI700633 | 2.33 | 0.00214 |
| Homo sapiens LOC349721 (LOC349721), mRNA | W61005 | 1.55 | 0.00214 |
| Homo sapiens mRNA; cDNA DKFZp586I1823 | AL080213 | 2.35 | 0.00214 |
| Homo sapiens transcribed sequences | N92548 | 1.95 | 0.00214 |
| Homo sapiens, clone IMAGE: 5288883, mRNA | W27075 | −1.76 | 0.00214 |
| hypocretin (orexin) receptor 2 | AF041245 | 1.56 | 0.00214 |
| hypothetical gene supported by AF038182; BC009203 | AF038182 | −2.01 | 0.00214 |
| hypothetical protein DKFZp566A1524 | AI138605 | −1.69 | 0.00214 |
| hypothetical protein FLJ10055 | AW052084 | 1.83 | 0.00381 |
| hypothetical protein FLJ90005 | W27419 | −1.57 | 0.00214 |
| hypothetical protein FLJ90798 | AL049949 | 1.98 | 0.00214 |
| hypothetical protein MGC35048 | AI674208 | 1.52 | 0.00214 |
| hypothetical protein MGC4614 | AF052106 | −1.61 | 0.00214 |
| hypothetical protein MGC5395 | M80899 | 1.93 | 0.00214 |
| hypoxia-inducible factor 1a | U22431 | 2.05 | 0.00512 |
| IDN3 protein | AB019494 | 1.64 | 0.00512 |
| inhibin, beta B (activin AB beta polypeptide) | M31682 | 1.78 | 0.00214 |
| inhibitor of DNA binding 4, | AL022726 | 2.32 | 0.00214 |
| inositol 1,4,5-trisphosphate 3-kinase A | X54938 | −2.46 | 0.00214 |
| inositol 1,4,5-trisphosphate 3-kinase B | X57206 | 1.93 | 0.00214 |
| inositol polyphosphate-4-phosphatase, type I, 107 kDa | AI955897 | −1.97 | 0.00214 |
| insulin receptor | X02160 | 1.59 | 0.00381 |
| integral membrane protein 2A | AL021786 | 1.72 | 0.00381 |
| integral membrane protein 2B | AA477898 | −1.53 | 0.00381 |
| integrin, alpha V | M14648 | 1.78 | 0.00214 |
| integrin, beta 1 | X07979 | 1.68 | 0.00214 |
| intercellular adhesion molecule 2 | X15606 | 1.57 | 0.00761 |
| intercellular adhesion molecule 2 | X15606 | 1.58 | 0.00992 |
| interferon regulatory factor 7 | U53831 | 1.89 | 0.00381 |
| interferon, gamma-inducible protein 16 | M63838 | 1.96 | 0.00512 |
| IQ motif containing GTPase activating protein 1 | L33075 | 1.76 | 0.00214 |
| KARP-1-binding protein | AB007939 | 1.64 | 0.00381 |
| KIAA0146 protein | M83667 | 1.86 | 0.00381 |
| KIAA0316 gene product | AB002314 | −2.09 | 0.00214 |
| KIAA0626 gene product | AB014526 | 1.59 | 0.00512 |
| KIAA0738 gene product | AB018281 | 1.55 | 0.00648 |
| KIAA0826 protein | AB020633 | 2.03 | 0.00761 |
| KIAA0828 protein | AB020635 | 1.7 | 0.00214 |
| KIAA0924 protein | AB023141 | 3.54 | 0.00512 |
| KIAA1155 protein | AA648931 | 1.51 | 0.00648 |
| kidney ankyrin repeat-containing protein | D79994 | 1.89 | 0.00214 |
| kinesin 2 60/70 kDa | L04733 | 1.7 | 0.00512 |
| kinesin family member 1B | AB011163 | −2.22 | 0.00214 |
| lamina-associated polypeptide 1B | AL050126 | 2.45 | 0.00992 |
| latent transforming growth factor beta binding protein 1 | M34057 | 1.79 | 0.00214 |
| leptin receptor | AW026535 | 1.69 | 0.00214 |
| leucine-rich repeats and immunoglobulin-like domains 1 | AL039458 | 1.97 | 0.00214 |
| likely ortholog of neuronally expressed calcium binding protein | W27472 | 1.62 | 0.00214 |
| LIM domain containing preferred translocation partner in lipoma | U49957 | 1.85 | 0.00512 |
| LIM protein (similar to rat protein kinase C-binding enigma) | AL049969 | 2.36 | 0.00214 |
| LIM protein (similar to rat protein kinase C-binding enigma) | AF061258 | 1.74 | 0.00214 |
| lipin I | D80010 | 1.55 | 0.00512 |
| lipopolysaccharide-induced TNF factor | AF010312 | 2.69 | 0.00648 |
| low density lipoprotein receptor-related protein 4 | AB011540 | 2.02 | 0.00214 |
| lysosomal-associated membrane protein 2 | U36336 | 2.34 | 0.00214 |
| lysosomal-associated membrane protein 2 | X77196 | 2.27 | 0.00214 |
| M13485 metallothionein I-B gene, exon 3 | M13485 | 1.61 | 0.00214 |
| MADS box transcription enhancer factor 2C | S57212 | −2.74 | 0.00214 |
| mal, T-cell differentiation protein | X76220 | 1.66 | 0.00214 |
| megalencephalic leukoencephalopathy with subcortical cysts 1 | D25217 | 1.61 | 0.00214 |
| metallothionein 1G | J03910 | 2.22 | 0.00512 |
| metallothionein 2A | R92331 | 1.72 | 0.00214 |
| metallothionein 2A | AI547258 | 1.51 | 0.00214 |
| metastasis suppressor 1 | AB007889 | 1.5 | 0.00761 |
| methionine adenosyltransferase II, alpha | X68836 | −2.08 | 0.00214 |
| microtubule-associated protein 1B | L06237 | −4.39 | 0.00214 |
| microtubule-associated protein 2 | U01828 | −4.17 | 0.00214 |
| microtubule-associated protein 2 | U01828 | −2.22 | 0.00214 |
| microtubule-associated protein 2 | U89330 | −2.13 | 0.00214 |
| microtubule-associated protein tau | J03778 | −2.31 | 0.00214 |
| microtubule-associated protein tau | X14474 | −2.33 | 0.00214 |
| mitochondrial ribosomal protein L28 | U19796 | −1.71 | 0.00214 |
| mitochondrial ribosomal protein S12 | Y11681 | −2.16 | 0.00214 |
| mitogen-activated protein kinase 1 | Z11695 | −1.85 | 0.00214 |
| mitogen-activated protein kinase 9 | U09759 | −1.73 | 0.00381 |
| mitogen-activated protein kinase kinase 4 | U17743 | −3.09 | 0.00214 |
| mitogen-activated protein kinase kinase kinase 5 | U67156 | 1.58 | 0.00214 |
| moesin | Z98946 | 1.66 | 0.00381 |
| monoamine oxidase A | AA420624 | 1.59 | 0.00214 |
| monocarboxylic acid transporters, member 1 | L31801 | 2.17 | 0.00214 |
| mRNA for ArgBPIB protein | X95677 | −2.59 | 0.00214 |
| mRNA for KIAA0631 protein | AB014531 | 2 | 0.00512 |
| mRNA full length insert cDNA clone EUROIMAGE 1630957 | AI018523 | 1.55 | 0.00214 |
| mRNA full length insert cDNA clone EUROIMAGE 1630957 | W28432 | 1.66 | 0.00879 |
| mRNA; cDNA DKFZp313P052 (from clone DKFZp313P052) | AA013087 | −3.82 | 0.00214 |
| mRNA; cDNA DKFZp566E0124 (from clone DKFZp566E0124) | AL050030 | −2.32 | 0.00214 |
| mRNA; cDNA DKFZp586B211 (from clone DKFZp586B211) | AL049423 | 1.88 | 0.00214 |
| muscleblind-like 2 (Drosophila) | AF061261 | −1.6 | 0.00214 |
| myeloid/lymphoid or mixed-lineage leukemia; translocated to, 2 | L13773 | 1.7 | 0.00761 |
| myosin regulatory light chain MRCL3 | X54304 | 1.66 | 0.00512 |
| myosin X | AB018342 | 2.13 | 0.00214 |
| nebulette | Y16241 | 1.52 | 0.00214 |
| N-ethylmaleimide-sensitive factor attachment protein, alpha | U39412 | −1.57 | 0.00512 |
| N-ethylmaleimide-sensitive factor attachment protein, gamma | U78107 | −2.23 | 0.00214 |
| neurexin 1 | AB011150 | −1.55 | 0.00214 |
| neuronal protein | W28770 | −1.96 | 0.00214 |
| neuronal/epithelial high affinity glutamate transporter, member 1 | U08989 | −1.55 | 0.00214 |
| Niemann-Pick disease, type C1 | AF002020 | 1.55 | 0.00879 |
| NIMA (never in mitosis gene a)-related kinase 7 | AL080111 | 1.98 | 0.00214 |
| NK2 transcription factor related, locus 2 (Drosophila) | AF019415 | 1.52 | 0.00512 |
| NK2 transcription factor related, locus 2 (Drosophila) | AF019415 | 1.77 | 0.00879 |
| N-myc downstream regulated gene 1 | D87953 | 1.55 | 0.00214 |
| nuclear factor (erythroid-derived 2)-like 2 | S74017 | 1.74 | 0.00214 |
| nuclear factor of kappa light polypeptide gene enhancer in B-cells | M69043 | 1.64 | 0.00214 |
| inhibitor, alpha | | | |
| nuclear receptor subfamily 1, group D, member 2 | D16815 | −2.95 | 0.00381 |
| nuclear transport factor 2 | X07315 | −1.89 | 0.00214 |
| nuclear transport factor 2-like export factor 2 | AL031387 | 1.78 | 0.00214 |
| OLF-1/EBF associated zinc finger gene | AB018303 | 2.04 | 0.00214 |
| oligodendrocyte lineage transcription factor 2 | U48250 | 1.55 | 0.00214 |
| oligodendrocyte lineage transcription factor 2 | U48250 | 1.68 | 0.00214 |
| O-linked N-acetylglucosamine (GlcNAc) transferase | U77413 | −1.55 | 0.00214 |
| oral-facial-digital syndrome 1 | Y15164 | 1.63 | 0.00992 |
| ornithine decarboxylase antizyme inhibitor | D88674 | −1.71 | 0.00214 |
| p21/Cdc42/Rac1-activated kinase 1 (STE20 homolog, yeast) | U24152 | −2.68 | 0.00214 |
| p21/Cdc42/Rac1-activated kinase 1 (STE20 homolog, yeast) | U24152 | −3 | 0.00214 |
| p8 protein (candidate of metastasis 1) | AI557295 | 2.7 | 0.00761 |
| palladin | AB023209 | 1.94 | 0.00214 |
| pantothenate kinase 3 | M55536 | −1.77 | 0.00214 |
| paraoxonase 2 | AF001601 | 1.58 | 0.00214 |
| PCF11p homolog | AB020631 | 1.77 | 0.00648 |
| peanut-like 2 (Drosophila) | AF035811 | 1.76 | 0.00761 |
| peripheral myelin protein 22 | D11428 | 1.72 | 0.00381 |
| peroxiredoxin 6 | D14662 | 1.7 | 0.00214 |
| peroxisome biogenesis factor 1 | AF026086 | 1.55 | 0.00879 |
| PHD finger protein 3 | D87685 | 1.84 | 0.00214 |
| phosphatidic acid phosphatase type 2A | AF014402 | 1.52 | 0.00648 |
| Phosphatidylinositol 3-Kinase P110, Beta | HG3254-HT3431 | −1.72 | 0.00214 |
| phosphatidylinositol binding clathrin assembly protein | U45976 | 1.57 | 0.00512 |
| phosphodiesterase 4D | U02882 | −1.92 | 0.00214 |
| phosphoinositide-3-kinase, class 2, alpha polypeptide | AL049998 | 1.84 | 0.00214 |
| phospholipase D3 | U60644 | −1.64 | 0.00214 |
| phosphotidylinositol transfer protein | D30036 | −1.58 | 0.00214 |
| phosphotidylinositol transfer protein, beta | D30037 | −2 | 0.00214 |
| platelet/endothelial cell adhesion molecule (CD31 antigen) | L34657 | 1.71 | 0.00214 |
| platelet/endothelial cell adhesion molecule-1 (PECAM-1) | L34657 | 2.23 | 0.00381 |
| pleckstrin homology domain containing, family B member 2 | AL120687 | −2 | 0.00879 |
| pleckstrin homology domain containing, family C member 1 | Z24725 | 1.64 | 0.00214 |
| pleckstrin homology domain containing, family E member 1 | AB011178 | 1.53 | 0.00879 |
| plexin B1 | AB007867 | 1.53 | 0.00214 |
| plexin C1 | AF030339 | 1.56 | 0.00761 |
| podocalyxin-like | U97519 | 1.64 | 0.00214 |
| poliovirus receptor-related 3 | AL050071 | −1.64 | 0.00214 |
| potassium inwardly-rectifying channel, subfamily J9 | U52152 | −2.22 | 0.00214 |
| procollagen-lysine, 2-oxoglutarate 5-dioxygenase 2 | U84573 | 2.43 | 0.00214 |
| proenkephalin | J00123 | −2.54 | 0.00381 |
| progestin induced protein | AF006010 | 1.56 | 0.00992 |
| pro-oncosis receptor inducing membrane injury gene | AL050161 | 1.87 | 0.00214 |
| protease, serine, 11 (IGF binding) | D87258 | 1.51 | 0.00214 |
| protein kinase C, beta 1 | X07109 | −2.89 | 0.00214 |
| protein kinase C, beta 1 | X06318 | −1.9 | 0.00214 |
| protein kinase C, gamma | Z15114 | −1.77 | 0.00214 |
| protein kinase C, zeta | Z15108 | −1.66 | 0.00214 |
| protein kinase, lysine deficient 1 | U00946 | 1.98 | 0.00214 |
| protein phosphatase 1E (PP2C domain containing) | AB028995 | −1.77 | 0.00214 |
| protein phosphatase 3 (calcineurin B, type I) | M30773 | −2.83 | 0.00214 |
| Protein Phosphatase Inhibitor Homolog | HG3570-HT3773 | −1.69 | 0.00214 |
| protein tyrosine phosphatase type IVA, member 2 | U14603 | 1.69 | 0.00214 |
| protein tyrosine phosphatase, receptor type, f polypeptide (PTPRF) | U22816 | 1.68 | 0.00381 |
| alpha 1 | | | |
| protein tyrosine phosphatase, receptor type, O | Z48541 | −3.8 | 0.00214 |
| protein-L-isoaspartate (D-aspartate) O-methyltransferase | D25547 | −2.7 | 0.00214 |
| proteolipid protein 1 | M54927 | 1.56 | 0.00214 |
| PTK9 protein tyrosine kinase 9 | U02680 | 1.84 | 0.00214 |
| putative transmembrane protein | U23070 | 1.62 | 0.00214 |
| quaking homolog, KH domain RNA binding (mouse) | AL031781 | 1.56 | 0.00214 |
| RAB1A, member RAS oncogene family | M28209 | −1.57 | 0.00648 |
| RAB3A, member RAS oncogene family | M28210 | −1.74 | 0.00214 |
| RAB5A, member RAS oncogene family | M28215 | −1.92 | 0.00214 |
| RAB6A, member RAS oncogene family | M28212 | −3.52 | 0.00214 |
| RAD23 homolog A (S. cerevisiae) | AD000092 | −1.51 | 0.00381 |
| RAN binding protein 9 | AF064606 | −1.73 | 0.00512 |
| RAP1B, member of RAS oncogene family | AL080212 | 1.7 | 0.00214 |
| RAP2A, member of RAS oncogene family | X12534 | −3.81 | 0.00214 |
| RAP2A, member of RAS oncogene family | X12534 | −4.13 | 0.00214 |
| Ras and Rab interactor 1 | L36463 | 1.53 | 0.00512 |
| Ras and Rab interactor 2 | AL049538 | 1.86 | 0.00214 |
| Ras association (RalGDS/AF-6) domain family 2 | D79990 | 1.92 | 0.00214 |
| Ras protein-specific guanine nucleotide-releasing factor 1 | S62035 | −4.04 | 0.00214 |
| Ras-Like Protein Tc4 | HG1112-HT1112 | −1.67 | 0.00214 |
| Ras-Like Protein Tc4 | HG1112-HT1112 | −1.63 | 0.00214 |
| Ras-Specific Guanine Nucleotide-Releasing Factor | HG2510-HT2606 | −2.41 | 0.00381 |
| Ras-Specific Guanine Nucleotide-Releasing Factor | HG2510-HT2606 | −4.74 | 0.00879 |
| receptor (calcitonin) activity modifying protein 1 | AJ001014 | 1.89 | 0.00214 |
| regucalcin (senescence marker protein-30) | D31815 | 1.7 | 0.00214 |
| regulator of G-protein signalling 19 interacting protein 1 | AF089816 | 1.59 | 0.00214 |
| regulator of G-protein signalling 20 | AF060877 | 2.28 | 0.00214 |
| regulator of G-protein signalling 4 | U27768 | −2.23 | 0.00214 |
| regulator of G-protein signalling 4 | AI267373 | −1.82 | 0.00214 |
| retinoblastoma binding protein 4 | X74262 | −1.75 | 0.00214 |
| retinoblastoma-associated protein 140 | AB029028 | 1.83 | 0.00761 |
| retinoblastoma-like 2 (p130) | X76061 | 1.65 | 0.00761 |
| retinol binding protein 4, plasma | X00129 | −1.52 | 0.00214 |
| Rho-related BTB domain containing 3 | AB020685 | 2.44 | 0.00214 |
| Rho-related BTB domain containing 3 | AB020685 | 2.78 | 0.00214 |
| ribonuclease, RNase A family, 1 (pancreatic) | D26129 | 1.7 | 0.00648 |
| ribosomal protein S21 | X79563 | 1.74 | 0.00214 |
| ribosomal protein S6 kinase, 90 kDa, polypeptide 3 | U08316 | −1.51 | 0.00214 |
| ryanodine receptor 1 (skeletal) | U48508 | 1.78 | 0.00214 |
| S100 calcium binding protein, beta (neural) | M59488 | 1.66 | 0.00214 |
| S-adenosylhomocysteine hydrolase-like 1 | R59606 | 1.59 | 0.00214 |
| S-adenosylhomocysteine hydrolase-like 1 | AI800578 | 1.63 | 0.00214 |
| sarcospan (Kras oncogene-associated gene) | N21470 | 1.96 | 0.00512 |
| secreted frizzled-related protein 1 | AF056087 | 1.88 | 0.00992 |
| selenoprotein P, plasma, 1 | Z11793 | 1.74 | 0.00214 |
| semaphorin 3B | U73167 | 1.82 | 0.00512 |
| septin 10 | AL049934 | 1.92 | 0.00381 |
| serine (or cysteine) proteinase inhibitor, clade H1, (collagen binding | D83174 | 1.67 | 0.00512 |
| protein 1) | | | |
| serine/threonine kinase 3 (STE20 homolog, yeast) | U26424 | 1.59 | 0.00648 |
| seryl-tRNA synthetase | X91257 | −1.54 | 0.00381 |
| SH3-domain GRB2-like endophilin B1 | AB007960 | 1.58 | 0.00214 |
| short-chain dehydrogenase/reductase 1 | AF061741 | 1.64 | 0.00214 |
| SMC5 structural maintenance of chromosomes 5-like 1 (yeast) | AB011166 | 1.76 | 0.00648 |
| sodium bicarbonate cotransporter, member 4 | AF007216 | 1.62 | 0.00214 |
| sodium channel, voltage-gated, type II, beta | AF049498 | −5.09 | 0.00214 |
| sodium-dependent inorganic phosphate cotransporter, member 7 | W26700 | −1.91 | 0.00214 |
| solute carrier family 14 (urea transporter), member 1 | U35735 | 2.44 | 0.00761 |
| somatostatin | AI636761 | −2.87 | 0.00214 |
| somatostatin | J00306 | −1.82 | 0.00214 |
| sortilin 1 | X98248 | −3.52 | 0.00214 |
| sperm specific antigen 2 | M61199 | 2.75 | 0.00648 |
| splicing factor proline/glutamine rich | W27050 | −1.52 | 0.00512 |
| splicing factor, arginine/serine-rich 16 (suppressor-of-white-apricot | AF042800 | 1.68 | 0.00879 |
| homolog) | | | |
| spondin 1, (f-spondin) extracellular matrix protein | AB018305 | 2.01 | 0.00214 |
| sterol carrier protein 2 | U11313 | 1.64 | 0.00214 |
| stomatin | X85116 | 1.56 | 0.00214 |
| stress 70 protein chaperone, microsome-associated, 60 kDa | U04735 | −1.76 | 0.00648 |
| SWI/SNF, actin dependent regulator of chromatin, subfamily a, | D26155 | −1.79 | 0.00214 |
| member 2 | | | |
| synapsin II | U40215 | −3.43 | 0.00214 |
| synaptophysin-like protein | X68194 | 1.8 | 0.00648 |
| synaptosomal-associated protein, 23 kDa | AJ011915 | 1.71 | 0.00512 |
| syndecan 2 | J04621 | 2.01 | 0.00214 |
| talin 1 | AB028950 | 1.52 | 0.00879 |
| Tax interaction protein 1 | U90913 | 1.87 | 0.00381 |
| TGFB-induced factor (TALE family homeobox) | X89750 | 1.95 | 0.00214 |
| thioredoxin interacting protein | S73591 | 1.84 | 0.00761 |
| thiosulfate sulfurtransferase (rhodanese) | D87292 | 1.52 | 0.00214 |
| Thy-1 cell surface antigen | AA704137 | −1.53 | 0.00214 |
| topoisomerase (DNA) II beta 180 kDa | M27504 | −1.72 | 0.00381 |
| topoisomerase I binding, arginine/serine-rich | U82939 | 1.6 | 0.00992 |
| transcription factor ZHX2 | AB020661 | 2.15 | 0.00214 |
| transcriptional co-activator with PDZ-binding motif (TAZ) | AL050107 | 2.58 | 0.00214 |
| transducer of ERBB2, 1 | D38305 | 1.72 | 0.00381 |
| transforming growth factor, beta receptor II (70/80 kDa) | D50683 | 1.77 | 0.00381 |
| transglutaminase 2 | M55153 | 2.83 | 0.00214 |
| trans-golgi network protein 2 | AF027516 | −2.19 | 0.00214 |
| translocase of inner mitochondrial membrane 17 homolog A (yeast) | X97544 | −2 | 0.00214 |
| transmembrane 4 superfamily member 1 | M90657 | 1.65 | 0.00381 |
| transmembrane 4 superfamily member 10 | AL049257 | 1.74 | 0.00214 |
| transmembrane 7 superfamily member 1 | AF027826 | 1.7 | 0.00214 |
| transmembrane trafficking protein | L40397 | 1.52 | 0.00761 |
| tripartite motif-containing 38 | U90547 | −1.6 | 0.00381 |
| trophoblast glycoprotein | Z29083 | −1.53 | 0.00214 |
| troponin T1, skeletal, slow | AJ011712 | 1.51 | 0.00512 |
| TSC-22-like | AJ133115 | 1.55 | 0.00214 |
| Tubulin, Alpha 1, Isoform 44 | HG2259-HT2348 | −1.69 | 0.00214 |
| tumor necrosis factor receptor superfamily, member 11b | AB008822 | 2.1 | 0.00512 |
| (osteoprotegerin) | | | |
| tumor necrosis factor receptor superfamily, member 21 | AF068868 | −1.8 | 0.00214 |
| tumor protein D52-like 2 | AF004430 | 1.61 | 0.00214 |
| tumor protein p53 binding protein, 2 | U58334 | 1.68 | 0.00214 |
| TWIST neighbor | AC004940 | 1.52 | 0.00214 |
| type I transmembrane C-type lectin receptor DCL-1 | D14664 | 1.68 | 0.00648 |
| Tyrosine Phosphatase 1, Non-Receptor, Alt. Splice 3 | HG3187-HT3366 | 2.22 | 0.00214 |
| ubiquinol--cytochrome-c reductase core protein I | H10776 | −1.99 | 0.00214 |
| ubiquitin carrier protein | M91670 | −1.62 | 0.00214 |
| ubiquitin carrier protein | M91670 | −1.66 | 0.00214 |
| ubiquitin-conjugating enzyme E2M (UBC12 homolog, yeast) | AF075599 | −1.63 | 0.00214 |
| Ubiquitin-Conjugating Enzyme Ubch5 | HG3344-HT3521 | −1.58 | 0.00214 |
| UDP-Gal:betaGlcNAc beta 1,3-galactosyltransferase, polypeptide 3 | Y15062 | −1.92 | 0.00214 |
| UDP-N-acetylglucosamine-2-epimerase/N-acetylmannosamine kinase | AJ238764 | 1.76 | 0.00761 |
| uracil-DNA glycosylase | Y09008 | 1.74 | 0.00512 |
| uroporphyrinogen III synthase (congenital erythropoietic porphyria) | J03824 | −1.51 | 0.00214 |
| vascular endothelial growth factor | AF022375 | 1.82 | 0.00512 |
| vascular endothelial growth factor B | U43368 | 1.51 | 0.00214 |
| VDACI pseudogene | AJ002428 | −2.44 | 0.00214 |
| v-erb-b2 erythroblastic leukemia viral oncogene homolog 3 (avian) | M34309 | 1.89 | 0.00214 |
| v-erb-b2 erythroblastic leukemia viral oncogene homolog 3 (avian) | M34309 | 2.29 | 0.00214 |
| vesicle amine transport protein 1 homolog (T californica) | U18009 | 1.68 | 0.00214 |
| vesicle-associated membrane protein 1 (synaptobrevin 1) | M36200 | −3.42 | 0.00214 |
| vesicle-associated membrane protein 3 (cellubrevin) | H93123 | 1.53 | 0.00214 |
| v-ets erythroblastosis virus E26 oncogene homolog 2 (avian) | J04102 | −1.68 | 0.00214 |
| vimentin | Z19554 | 1.88 | 0.00214 |
| von Willebrand factor | M10321 | 2.09 | 0.00214 |
| v-yes-1 Yamaguchi sarcoma viral oncogene homolog 1 | M15990 | 1.7 | 0.00214 |
| Wolfram syndrome 1 (wolframin) | AF084481 | 1.66 | 0.00214 |
| zinc finger and BTB domain containing 1 | AI970189 | 1.82 | 0.00879 |
| zinc finger homeobox 1b | AB011141 | 2.07 | 0.00214 |
| zinc finger protein 238 | U38896 | −4.59 | 0.00214 |
| Zinc Finger Protein, Kruppel-Like | HG3635-HT3845 | −1.63 | 0.00761 |

### Example 4

### Ageing and Vunerable Genes

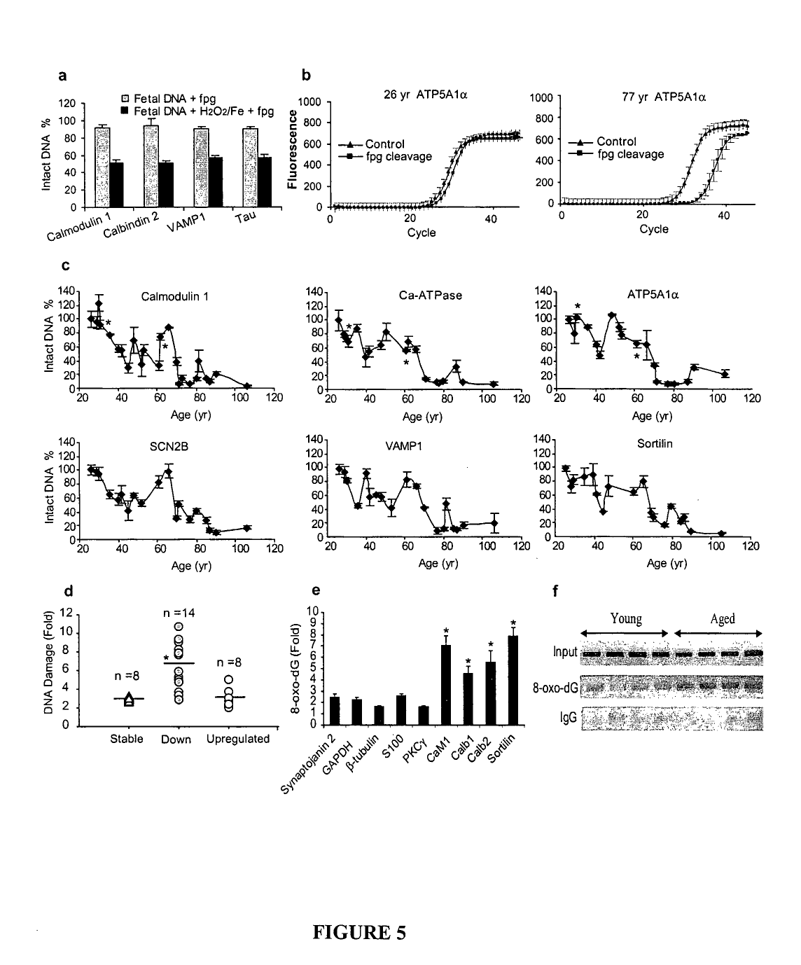

The pronounced downregulation of a defined gene cluster followed by induction of antioxidant and DNA repair genes led us to hypothesize that oxidative DNA damage might target specific genes. Promoter regions may be especially vulnerable, as they contain (G+C)-rich sequences that are highly sensitive to oxidative DNA damage and are not protected by transcription-coupled repair (Tu et al. (1996) EMBO J. 15, 675-683). To explore this hypothesis, an assay was devised to detect DNA damage in specific gene sequences. Genomic DNA was isolated under conditions that prevent in vitro oxidation, and then cleaved with formamidopyrimidine-DNA glycosylase (fpg), which is an N-glycosidase and AP-lyase that selectively releases damaged bases from DNA, predominantly affecting the major oxidation product 8-oxoguanine (Tchou et al. (1994) J. Biol. Chem. 269, 15318-15324. Fpg creates a single-strand break at the apurinic site, rendering it resistant to PCR amplification. Hence, DNA damage to specific sequences can be determined from the ratio of intact PCR products in cleaved versus uncleaved DNA using quantitative PCR. This assay was used to assess damage in genomic DNA from fetal human cortex. Fetal cortical DNA did not show significant oxidative DNA damage in the 1-kb upstream promoter regions of several genes that show age-related changes in expression in the adult brain (FIG. 5A).

To determine whether DNA damage increases in the ageing human cortex, and whether there is a predilection for specific genes, the promoters of 30 different genes were examined. Each of these genes showed increased promoter DNA damage in the aged cortex. DNA damage appeared in many genes after age 40, and was most pronounced in all genes after age 70 (FIGS. 5B and 5C). DNA damage also occurred in the exons of these genes with a similar time course, but to a lesser extent than in the promoter regions (data not shown). Biopsy samples of human cortex from individuals who underwent elective neurosurgical procedures showed a similar pattern of age-related DNA damage as postmortem samples (FIG. 5C, asterisks). Thus, DNA damage is pervasive in the ageing human cortex.

To examine whether gene downregulation in the ageing brain was associated with accelerated DNA damage, the increase in promoter DNA damage, indicated by the reduction in intact DNA in individuals over 70 years old, was examined. This index of DNA damage was compared for genes that were downregulated, upregulated or stably expressed in the aged cortex. Stably expressed and upregulated genes showed a narrow range of promoter DNA damage in aged cortex (FIG. 5D). In contrast, most of the age-downregulated genes showed significantly greater DNA damage in the aged cortex (P<0.001) (FIG. 5D). These results were confirmed by independently assaying 8-oxoguanine through cleavage of genomic DNA with the 8-oxoguanine-specific N-glycosylase human OGG1. 8-oxoguanine levels were markedly increased in the promoters of most of the age-downregulated genes examined (FIG. 5E). Chromatin immunoprecipitation of the calmodulin 1 promoter with a monoclonal antibody to 8-oxoguanine confirmed an approximately eightfold increase in 8-oxoguanine in aged cortical samples (FIG. 5F). Thus, accelerated DNA damage is associated with reduced gene expression in the aged human cortex.

### Example 5

### DNA Damage and Gene Expression

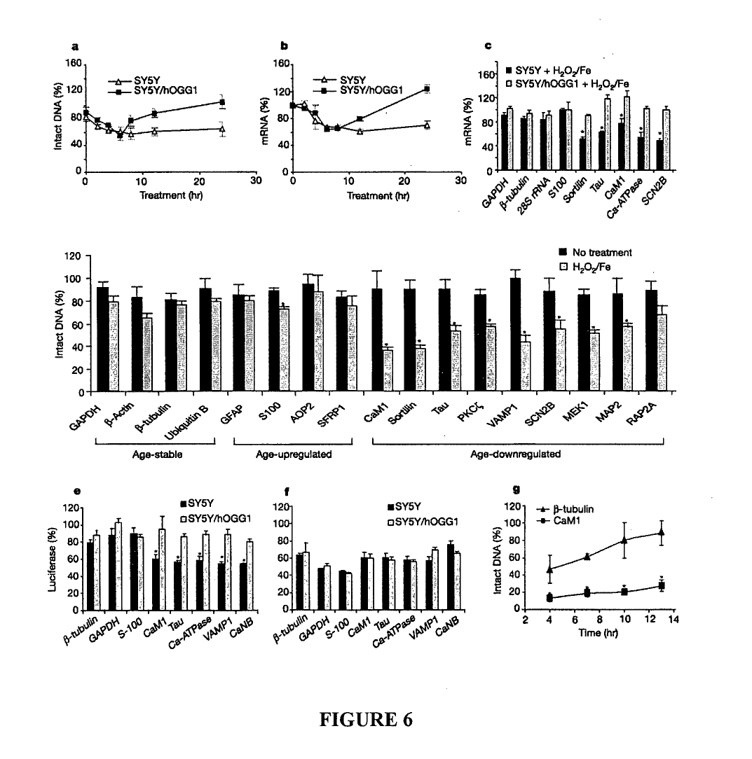

To obtain greater insight into the effects of DNA damage on gene expression, human neuroblastoma SH-SY5Y cell lines were produced that stably overexpress the base-excision repair enzyme human OGG1 (Lindahl, Cold Spring Harb. Symp. (2000) Quant. Biol. 65, 127-133 and Bruner (2000) Cold Spring Harb. Symp. Quant. Biol. 65, 103-111). FIG. 6 shows promoters of age-downregulated genes show increased vulnerability to oxidative DNA damage. (See FIGS. 6A and 6B) Human neuroblastoma SH-SY5Y cells were incubated with H₂O₂/FeCl₂ for the indicated time intervals to induce oxidative DNA damage. DNA damage (FIG. 6A) and mRNA expression (FIG. 6B) of the tau gene were determined in cells that overexpress the DNA repair enzyme human OGG1 (SY5Y/hOGG1) or the empty pcDNA3 vector (SY5Y). DNA damage was determined by the FPG cleavage/PCR-based assay. FIG. 6C, mRNA levels of age-downregulated genes are selectively reduced by oxidative stress and restored by human OGG1. mRNA levels are expressed as percentage in the presence versus absence of H₂O₂/FeCl₂ and represent the mean ±s.e.m.; n=4. Asterisk indicates P<0.05 relative to no treatment by ANOVA with post-hoc Student-Newman—Keuls test. FIG. 6D, Increased vulnerability to oxidative DNA damage in promoters of age-downregulated genes. Human cortical neuronal cultures were incubated in the presence or absence of 100 μM H₂O₂/20 μM FeCl₂ for 12 hours and promoter DNA damage was assayed. Values represent the mean ±s.d.; n=3. Asterisks indicate P<0.05 relative to no treatment P<0.001 for the group of age-downregulated genes relative to age-stable or age-upregulated genes. FIG. 6E, Reduced transcriptional activity of promoters of age-down-regulated genes following oxidative DNA damage. Luciferase reporter plasmids derived from the promoters of age-downregulated genes (calmodulin 1 (CaM1), tau, Ca-ATPase, VAMP1/synaptobrevin, and calcineurin B (CaNB)) and genes without reduced expression (β-tubulin, GAPDH and S100) were incubated in the absence or presence of 100 μM H₂O₂ for one hour in vitro, and then transfected into SH-SY5Y or SH-SY5Y/human OGG1 cells. Shown is luciferase activity of the damaged reporter expressed as percentage of the activity of the undamaged reporter after 16 h. Values represent the mean ±s.d.; n=4. Asterisk indicates P<0.05 for SH-SY5Y relative to SH-SY5Y/human OGG1. FIG. 6F, Ultraviolet damage does not discriminate between promoters of age-stable and age-downregulated genes. FIG. 6G, DNA damage and repair of the β-tubulin and calmodulin 1 (CaM1) promoters. Reporter plasmids damaged in vitro by H₂O₂ were transfected and DNA damage was determined within each promoter sequence at increasing time intervals. Values are expressed relative to the transfected undamaged reporter, and represent the mean ±s.d.; n=3. Asterisks indicate P<0.05 relative to β-tubulin.

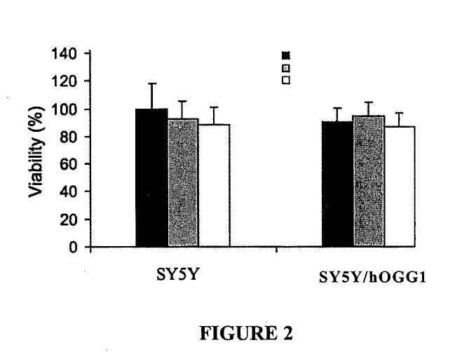

Oxidative DNA damage was induced by incubating SH-SY5Y cells with H₂O₂ and FeCl₂, resulting in rapid DNA damage to the promoter of the tau gene, followed by slow and incomplete DNA repair (FIG. 6A). In contrast, human OGG1-overexpressing SH-SY5Y cells showed augmented DNA repair with complete restoration of intact DNA (FIG. 6A). Tau mRNA expression was also reduced by oxidative stress, but was completely restored by overexpression of human OGG1 (FIG. 6B). Cell viability was not significantly affected by the mild oxidative stress treatment or by overexpression of human OGG1 (FIG. 2). Thus, oxidative DNA damage can reduce gene expression.

Endogenous mRNA levels of a number of age-downregulated genes (tau, calmodulin 1, Ca-ATPase, sortilin and the sodium channel 2β) were significantly reduced by mild oxidative stress in SH-SY5Y cells, and restored by human OGG1 (FIG. 6B). In contrast, mRNA levels of genes that are not reduced in the ageing cortex (β-tubulin, GAPDH, S100 and 28S RNA) were not significantly affected (FIG. 6C). Thus, mRNA levels of some age-downregulated genes are highly sensitive to oxidative DNA damage.

A larger number of promoters from age-downregulated and age-stable genes were surveyed to assess vulnerability to DNA damage in cultured human neurons. After pro-oxidative stress, the promoters of four age-stable and four age-upregulated genes showed minimal declines in the level of intact DNA (FIG. 6D). In contrast, eight of nine age-downregulated promoters showed significantly increased DNA damage. Thus, promoters of age-downregulated genes show increased vulnerability to oxidative DNA damage.

### Example 6

### DNA Repair and Promoter Vunerability

To determine whether reduced DNA repair contributes to promoter vulnerability, we cloned the promoters in luciferase reporter plasmids and performed a host cell reactivation assay (Athas et al. (1991) Cancer Res. 51: 5786-5793). Promoter reporter plasmids were damaged in vitro by either treatment with H₂O₂ or exposure to ultraviolet light, and then transfected into SH-SY5Y cells, together with an undamaged renilla luciferase control plasmid. Activation of H₂O₂-damaged reporters, an indicator of base-excision repair, was significantly reduced for reporters derived from the promoters of age-downregulated genes relative to reporters derived from age-stable genes (FIG. 6E). Reporter activity was restored by the base-excision repair enzyme human OGG1. In contrast, activation of ultraviolet-damaged reporters was not significantly different for the two promoter categories, and was not affected by human OGG1 (FIG. 6F). Differential promoter damage was confirmed using the FPG cleavage/PCR-based assay. The calmodulin 1 promoter showed more DNA damage than the β-tubulin promoter, and was repaired very slowly (FIG. 6G). In contrast, the β-tubulin promoter was repaired much more rapidly (P=0.007). Thus, both increased initial damage and reduced base-excision repair may contribute to oxidative DNA damage in age-downregulated genes.

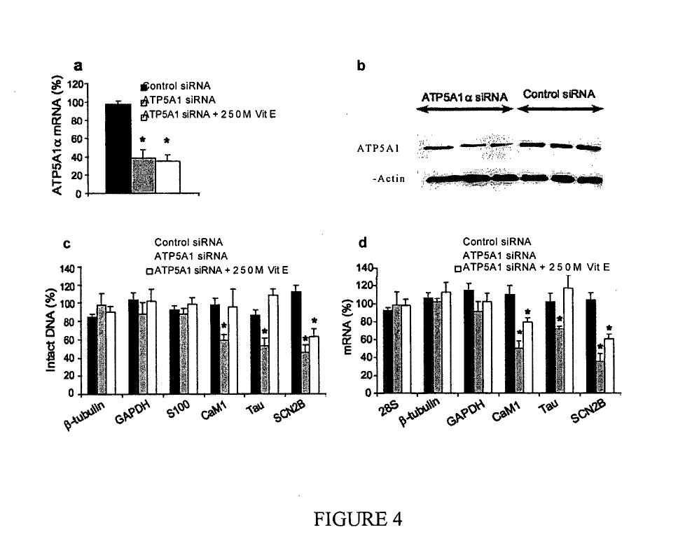

One factor that may predispose to DNA damage in the aged cortex is impaired mitochondrial function. Expression of the α subunit of the mitochondrial F1 ATP synthase, which couples oxidative phosphorylation to ATP synthesis, was significantly reduced in the aged human cortex (Table 1 and FIG. 3A). siRNA was used to reduce expression of F1 ATP synthase a mRNA and protein by 2.5-fold in SH-SY5Y cells (FIGS. 4A and 4B), approximating the reduction detected in aged human cortex. This resulted in a 24±1% reduction in cellular ATP levels (mean ±s.d.; n=12; P=0.014), but did not affect overall cell viability or induce apoptosis. ATP synthase a small interfering RNA significantly increased promoter DNA damage in age-downregulated genes, and reduced mRNA levels (FIGS. 4C and 4D). Another siRNA (topoisomerase IIβ) and random 21-mer oligonucleotide controls had no significant effects (FIGS. 4A-D). DNA damage induced by knockdown of F1 ATP synthase α was partially reversed by the antioxidant vitamin E (FIGS. 4C and 4D). Thus, impaired mitochondrial function can lead to nuclear DNA damage.

Taken together, these findings suggest that accelerated DNA damage may contribute to reduced gene expression in the human brain after age 40. The cluster of age-downregulated genes includes many genes that play integral roles in synaptic plasticity, including NMDA and AMPA receptor function, calcium-mediated signalling, and synaptic vesicle release and recycling (Kandel (2001) Science 294, 1030-1038 and Malinow, et al. (2002) Annu. Rev. Neurosci. 25, 103-126). In addition, the reduced expression of key calcium-binding and homeostatic genes in the aged cortex could compromise intraneuronal calcium homeostasis, as observed in studies of ageing rodent neurons and may increase neuronal vulnerability to toxic insults (Landfield, et al. (1984) Science 226, 1089-1092). These findings also provide support for the concept of ongoing oxidative stress in the ageing human cortex as a variety of oxidative stress response and repair genes were induced (Longo, et al. (2003) Science 299, 1342-1346; Hekimi, et al. (2003) Science 299, 1346-1351; and Hasty et al. (2003) Science 299: 1355-1359). Similar stress response genes are induced in the ageing mouse and rat brain (Lee, et al. (2000) Nature Genet. 25, 294-297 and Jiang, et al. (2001), Proc. Natl Acad. Sci. USA 98, 1930-1934) and modulate ageing in C. elegans (Murphy, et al. (2003) Nature 424, 277-283). Thus, genome damage may compromise systems that subserve synaptic function and neuronal survival, leading to compensatory s tress responses in the aged cortex.

Selective DNA damage to gene promoter sequences is a potential mechanism whereby the expression of specific genes could register the passage of time. Vulnerable DNA sequences appear to show increased initial DNA damage as well as reduced base excision repair. It will be of interest to define the specific vulnerable sequence motifs and their target transcription factors (Ghosh, et al. (1999) Nucleic Acids Res. 27, 3213-3218; and Kyng, et al. (2003) Proc. Natl. Acad. Sci. USA 100, 12259-12265). A previous study showed that there is also substantial oxidative damage to mitochondrial DNA in the ageing human brain (Mecocci, et al. (1993) Ann. Neurol. 34, 609-616. These findings suggest that impaired mitochondrial function may contribute to the damage of vulnerable genes in the ageing cortex by increasing reactive oxygen species or by reducing ATP required for DNA repair.

When ageing begins and what triggers its onset is one of the major conundrums of biology. The young adult and extreme aged human populations are relatively homogeneous in their gene expression patterns in prefrontal cortex. However, the middle age population between 40 and 70 years of age exhibits much greater heterogeneity. Thus, individuals may diverge in their rates of ageing as they transit through middle age, approaching a state of ‘old age’ at different rates. It will be of interest to investigate this relationship in different populations and demographic groups. A recent report suggests that ageing in C. elegans and Drosophila is characterized by similar changes in orthologous mitochondrial and DNA repair genes, and that the pattern is established in early adulthood (McCarroll et al. (2004) Nature Genet. 36, 197-204. Thus, measures to protect the genome early in adult life may influence the rate of subsequent functional decline and the vulnerability of the brain to age-related neurodegenerative diseases.

What is claimed is:

1. A method of assessing oxidative stress in a subject, comprising;
obtaining a sample of nucleic acid from the subject;
measuring a level of expression associated with at least one metal ion homeostasis gene in the sample; and
comparing the measured level with at least one reference value, whereby a high level of expression indicates a heightened level of oxidative stress in the individual.

2. The method of claim 1, wherein the metal ion homeostasis gene is selected from the group consisting of a metallothionein 1G gene, a metallothionein 1B gene, a metallothionein 2A gene, a haem binding protein 2 gene, and a haemoglobin gene.

3. The method of claim 1, wherein the sample is isolated from a fluid selected from the group consisting of blood, serum, and cerebrospinal fluid.

4. The method of claim 1, wherein the reference value is established from at least one subject under the age of 30 years old.

5. A method of assessing an age-related condition in a subject, comprising;
obtaining a sample of nucleic acid from the subject;
measuring a level of expression associated with at least one metal ion homeostasis gene and at least one hormone gene; and
comparing the measured level with at least one reference value, whereby high levels of expression of the metal ion homeostasis gene and the hormone gene indicate an age-related condition in the subject.

6. The method of claim 5, wherein metal ion homeostasis gene is selected from the group consisting of a metallothionein 1G gene, a metallothionein 1B gene, a metallothionein 2A gene, a haem binding protein 2 gene, and a haemoglobin gene.

7. The method of claim 5, wherein the hormone gene is selected from the group consisting of an insulin receptor gene, an orexin receptor gene, a vascular endothelial growth factor gene, and a secreted frizzled related protein-1 gene.

8. The method of claim 5, wherein the hormone gene is an orexin receptor gene.

9. The method of claim 5, wherein the hormone gene is a secreted frizzled related protein-1 gene.

10. The method of claim 5, further comprising measuring the level of expression of hormone gene selected from the group consisting of a proenkephalin gene, a somatostatin gene, and a cholecystokinin B receptor gene, wherein a low level of expression of the hormone gene compared to at least one reference value indicates an age-related condition in the subject.

11. The method of claim 5, wherein the hormone gene is a proenkephalin gene.

12. The method of claim 5, wherein the sample is isolated from a fluid selected from the group consisting of blood, serum, and cerebrospinal fluid.

13. The method of claim 5, further comprising measuring the level of expression of at least one calcium homeostasis gene selected from the group consisting of calmodulin 1, CaM kinase II, and calbindin 1, wherein a low level of expression of the calcium hormone gene indicates an age-related condition in the subject.

14. The method of claim 5, further comprising measuring the level of expression of a calmodulin 1 gene, wherein a low level of expression of the calmodulin 1 gene indicates an age-related condition in the subject.

15. The method of claim 5, wherein the reference value is established from a subject under the age of 30 years old.

16. The method of claim 5, wherein the age-related disease is selected from the group consisting of Alzheimer's disease, Huntington's disease, Parkinson's disease, senile dementia, akathesia, amnesia, bipolar disorder, catatonia, cerebrovascular disease Creutzfeldt-Jakob disease, dementia, depression, tardive dyskinesia, dystonias, epilepsy, multiple sclerosis, neuralgias, neurofibromatosis, neuropathies, and schizophrenia.
